(19) United States
(12) Patent Application Publication
(10) Pub. No.: US 2009/0301472 A1
(43) Pub. Date: Dec. 10, 2009

(54) AEROSOL DELIVERY SYSTEMS AND METHODS

(76) Inventors: Matthew H.J. Kim, Norcross, GA (US); Stephanie R.S. Anderson, Atlanta, GA (US); Michael Axelrod, Roswell, GA (US); James E. Maciariello, Raleigh, NC (US); Joseph L. Kapushion, Erie, CO (US); Doug E. Zaugg, Longmont, CO (US); Andrew S. Sawyer, Denver, CO (US)

Correspondence Address: Ballard Spahr LLP, SUITE 1000, 999 PEACHTREE STREET, ATLANTA, GA, 30309-3915, US

(21) Appl. No.: 12/069,430

(22) Filed: Feb. 8, 2008

Related U.S. Application Data
(60) Provisional application No. 60/900,627, filed on Feb. 8, 2007.
(60) Provisional application No. 60/905,169, filed on Mar. 5, 2007.

(51) Int. Cl. A61M 11/00

(52) U.S. Cl. 128/200.16; 128/200.23; 128/200.18

(57) ABSTRACT

Methods, systems and/or devices for generating and aerosol from one or more composition are provided herein.

### CROSS REFERENCE TO RELATED APPLICATIONS

This application claims benefit of U.S. Provisional Application Ser. No. 60/900,627, filed Feb. 8, 2007 and U.S. Provisional Application Ser. No. 60/905,169, filed Mar. 5, 2007, which applications are hereby incorporated by reference in their entirety.

### BACKGROUND

Needles and syringes have posed a variety of problems for administration of agents to subjects, including, but not limited to, injection safety, needle stick injury, disposal problems, transmission of blood borne diseases, and needle shortages. The replacement of needles and syringes as a delivery vehicle for agents has the potential for tremendous cost savings, increased safety and reduction of biomedical wastes.

Aerosol generation can also be a valuable method for industrial and other commercial and consumer applications.

### SUMMARY

The present disclosure concerns methods, systems and/or devices for generating and aerosol from one or more composition.

It is to be understood that both the foregoing general description and the following detailed description are exemplary and explanatory only and are not restrictive of the invention, as claimed.

### BRIEF DESCRIPTION OF THE DRAWINGS

The accompanying drawings illustrate one or more embodiments of the invention and, together with the written description, serve to explain the principles of the invention.

FIGS. 1A and 1B are side views, shown partially in section, of an aerosol delivery device, according to one embodiment.

FIG. 2 is a side view of an aerosol delivery device, according to another embodiment.

FIGS. 3A and 3B are side views, shown partially in section, of an aerosol delivery device, according to yet another embodiment.

FIGS. 4A and 4B are front elevation and cross-sectional views, respectively, of a removable and disposable aerosolization element for an aerosol delivery device, according to one embodiment.

FIG. 5 is cross-sectional view of an embodiment of a capillary feed aerosolization element for an aerosol delivery device.

FIG. 6 is a front elevation view of another embodiment of an aerosolization element for an aerosol delivery device.

FIGS. 7A and 7B are front elevation and cross-sectional views, respectively, of another embodiment of an aerosolization element for an aerosol delivery device.

FIGS. 8A and 8B are front elevation and cross-sectional views, respectively, of another embodiment of an aerosolization element that is used to store and mix two liquid components. FIG. 8C is a cross-sectional view similar to FIG. 8B, showing the aerosolization element after the liquid components are mixed together to form an agent to be aerosolized.

FIG. 9A is a cross-sectional view of another embodiment of an aerosolization element that contains a liquid component separated from a dry component. FIG. 9B is a cross-sectional view similar to FIG. 9A, showing the aerosolization element after the liquid component and dry component are mixed together to form an agent to be aerosolized.

FIG. 10A is a front elevation view of another embodiment of an aerosolization element for an aerosol delivery device. FIG. 10B is a cross-sectional view taken along line 10B-10B of FIG. 10A. FIG. 10C is a magnified cross-sectional view of a portion of the aerosolization element.

FIG. 11 is an enlarged cross-sectional view of a patient interface shown being used with the aerosol delivery device of FIGS. 1A and 1B.

FIG. 12 is an enlarged cross-sectional view of another embodiment of a patient interface shown being used with the aerosol delivery device of FIGS. 1A and 1B.

FIG. 13 is an enlarged cross-sectional view of another embodiment of a patient interface shown being used with the aerosol delivery device of FIGS. 1A and 1B.

FIG. 14 is an enlarged cross-sectional view of another embodiment of a patient interface shown being used with the aerosol delivery device of FIGS. 1A and 1B.

FIG. 15 is an enlarged cross-sectional view of another embodiment of a patient interface shown being used with the aerosol delivery device of FIGS. 1A and 1B.

FIG. 16 is an enlarged cross-sectional view of a patient interface, according to another embodiment, shown being used with the aerosol delivery device of FIGS. 1A and 1B and having a plurality of internal baffles in the flow path to the patient.

FIGS. 17A and 17B are enlarged cross-sectional views of a patient interface, according to another embodiment, showing the operation of a one-way valve in the patient interface permitting flow from the aerosol delivery device to a patient, but inhibiting flow in the opposite direction.

FIGS. 18A and 18B are enlarged cross-sectional views of a patient interface, according to another embodiment, showing the operation of a one-way, duckbill valve in the patient interface.

FIGS. 19A and 19B are enlarged cross-sectional views of a patient interface having a one-way valve, according to another embodiment.

FIGS. 20A and 20B are enlarged cross-sectional views of a patient interface having a one-way valve, according to another embodiment.

FIG. 21A is a side elevation view of a piezoelectric actuator for an aerosol delivery device and a heat sink coupled to the actuator. FIG. 21B is an end view of the heat sink shown in FIG. 21A.

FIGS. 22A, 22B, and 22C are front elevation, cross-sectional, and exploded views of an aerosolization element, according to another embodiment.

FIG. 23 is a cross-sectional view of an alternative embodiment of an aerosol delivery device.

FIGS. 24A and 24B are front and side elevation views, respectively, of components of the aerosol delivery device shown in FIG. 23. FIG. 24C is a cross-sectional view taken along line 24C-24C of FIG. 24A.

FIG. 25A is an exploded perspective view of an exemplary removable aerosolization device.

FIG. 25B is a perspective view of the exemplary removable aerosolization device of FIG. 25A.

FIG. 25C is a perspective view of the exemplary removable aerosolization device with its source of agent removed from the receptacle of the body.

FIG. 25D shows a perspective view and a cross-sectional view, respectively, of the exemplary removable aerosolization device of FIG. 25A.

FIG. 26 is a schematic illustration of an exemplary biasable element or plate and a cross-sectional view, respectively, of the biasable element taken along line A-A.

FIG. 27 is a partial, schematic cross-sectional view of a removable aerosolization device operatively seated on portions of an exemplary aerosol delivery device.

FIG. 28 is a schematic illustration showing exemplary aspects of a removable aerosolization device and aerosol delivery device.

FIG. 29 is a schematic illustration showing exemplary aspects of a removable aerosolization device and aerosol delivery device.

FIG. 30 is a schematic illustration showing exemplary aspects of a removable aerosolization device and aerosol delivery device.

FIG. 31 is a schematic illustration showing exemplary aspects of a removable aerosolization device and aerosol delivery device.

FIG. 32 is a schematic illustration showing exemplary aspects of a removable aerosolization device and aerosol delivery device.

FIG. 33 is a schematic illustration showing exemplary aspects of a removable aerosolization device and aerosol delivery device.

FIG. 34 is a schematic illustration showing exemplary aspects of a removable aerosolization device and aerosol delivery device.

FIG. 35 is a schematic illustration showing exemplary means of introducing agent into a removable aerosolization element.

FIG. 36 is a schematic illustration showing exemplary means of introducing agent into a removable aerosolization element.

FIG. 37 is a schematic illustration showing exemplary means of introducing agent into a removable aerosolization element.

FIG. 38 is a schematic illustration showing exemplary means of introducing agent into a removable aerosolization element.

FIG. 39 is a schematic illustration showing exemplary means of introducing agent into a removable aerosolization element.

FIG. 40 is a partial, schematic illustration showing exemplary aspects of a removable aerosolization element and an aerosol delivery device.

### DETAILED DESCRIPTION

As used herein, the singular forms “a,” “an,” and “the” refer to one or more than one, unless the context clearly dictates otherwise.

As used herein, the term “includes” means “comprises.”

Agents, as used herein, comprise any composition or component thereof that can aerosolized. In one aspect, an agent is any agent that can be administered to the pulmonary system of a subject. For example, an agent can be administered to living organisms for an effect in the treated organism. Such agents include live and killed organisms for vaccination, immunogens, immune activators or suppressors, chemotherapeutics, pharmaceuticals, nucleic acids, insulin, hormones, antibodies and fragments thereof, receptors, proteins, carbohydrates, fats, nutrients, anesthetics, narcotics, and pain relievers. Agents can also agents used for consumer and industrial applications. For example, an industrial use includes, but is not limited to, application of an even and fine coat for silicon wafers. Other commercial or consumer applications can include agents or compositions used in humidifiers, printers, fragrance, repellants, paint, repellants, skin & beauty care, etc.

Thus the systems, methods and devices described herein can be used for medical type applications where a therapeutic composition is to be administered to a subject. In other examples, the methods, systems and devices and components thereof can be used in any industrial application where an aerosol is produced from a composition. In other examples, the methods, systems and devices can be used in commercial or consumer applications where an aerosol is produced from a composition.

Certain embodiments of the present system utilize an actuator coupled to a disposable aerosolizing element that aerosolizes an agent for delivery to a patient when acted upon by the actuator. The aerosolizing element prevents the agent from contacting the actuator and other non-disposable components of the system so that little or no cleaning or maintenance is required. The system therefore is well suited for use by less-trained personnel in high-workload applications, such as mass vaccination campaigns.

The present system also can include an aerosolization rate monitor that monitors the rate at which an agent is being aerosolized and provides feedback to the user to ensure that the proper dose is being administered. For example, the system can include an indicator light that illuminates or flashes if the aerosolization rate is outside an acceptable range.

Exemplary methods of the present disclosure comprise delivery of agents such as therapeutic compositions. In other exemplary methods the disclosed systems, devices and/or methods can be used to deliver non-therapeutic agents. For example, consumer and industrial applications can be made using the disclosed systems, devices and/or methods. In these examples, an aerosol can be delivered into a subject, or into an industrial, commercial, or consumer system.

The methods of the present disclosure comprise delivery of compositions via aerosol administration. The present disclosure contemplates the use of any composition that can be delivered via aerosol administration. In one aspect, a composition comprises a vaccine. Particularly preferred vaccination compositions are those for measles, mumps and rubella. Thus, compositions may comprise measles vaccine, mumps vaccine, rubella vaccine and combinations and mixtures such as measles and mumps, rubella and mumps, measles and rubella, and measles, mumps and rubella. Any administered compositions can further comprise pharmaceutical or formulation components such as those known in the art, including, but not limited to, diluents, compounding agents, surfactants, and agents to maintain sterility.

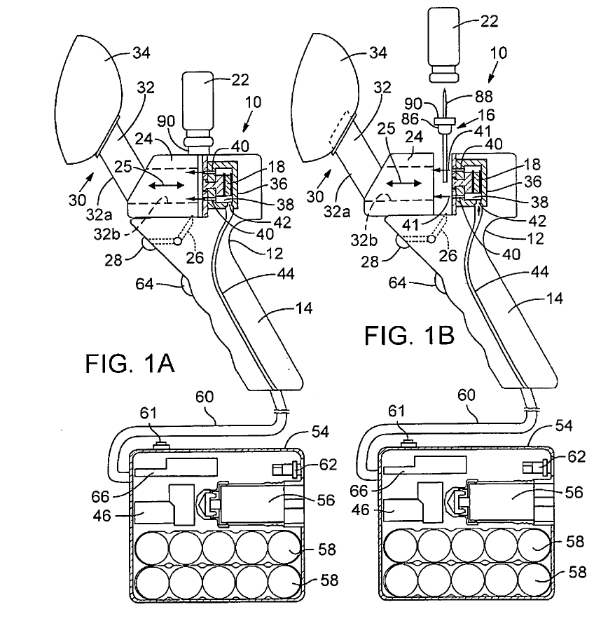

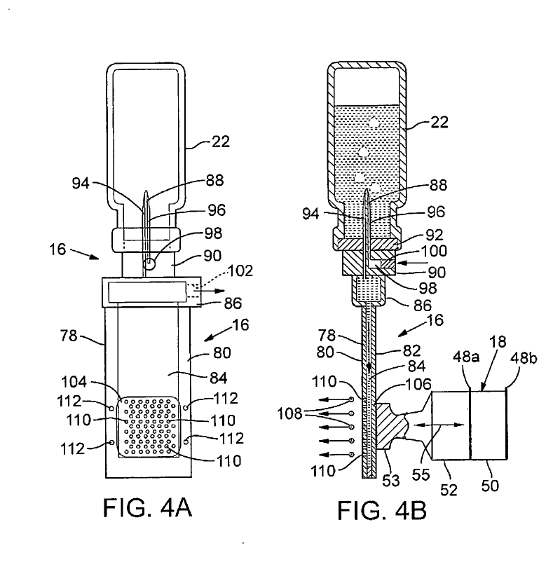

FIGS. 1A and 1B depict an aerosol delivery device 10, according to one embodiment. The aerosol delivery device 10, includes a body, or housing 12 formed with a handle portion 14 shaped to be held in a user's hand. The housing 12 in the illustrated embodiment houses a removable aerosolizing element 16, an actuator 18, and an air manifold 36 substantially surrounding the actuator 18. The illustrated aerosolizing element 16 can be directly coupled to a vial 22 containing an agent to be administered. As described in detail below, the aerosolizing element 16 receives the agent from the vial 22 and expels aerosol droplets through orifices 110 (FIG. 4A) for delivery upon activation of the actuator 18.

The housing 12 is formed with a movable front portion 24 that is mounted for sliding movement in the directions indicated by double-headed arrow 25 between a closed position (as shown in FIG. 1A) and an open position (as shown in FIG. 1B) to allow access to the aerosolizing element 16.

When the front portion 24 is in the closed position, the aerosolizing element 16 is held firmly in place between the front portion and the actuator 18. A latch mechanism 26 and a latch button 28 can be provided to releasably retain the front portion 24 in the closed position. Depressing the latch button 28 removes the latch mechanism 26 from engagement with the front portion 24 so that it can be moved to the open position. The front portion 24 desirably is adapted to be completely removable from the housing 12 for ease of cleaning. While the illustrated front portion 24 is mounted for sliding movement relative to the housing 12 as shown, any other detachable connection can be used to mount the front portion 24 to the housing (e.g., adhesives, snap fittings, etc.).

Coupled to the housing 12 is an optional patient interface 30 for delivering an aerosolized agent to a patient according to certain embodiments. The illustrated patient interface 30 includes a generally cylindrical extension portion 32 connected to the movable portion 24 and a face mask 34 or other user interface mounted to the upper end of the extension portion 32. The mask 34 is mounted to the extension portion 32 in a removable manner so that the mask can be easily removed and replaced with a new mask for another patient or subject. The extension portion 32 includes a first portion 32a extending through the front portion 24 of the housing 12 and a second portion 32b that extends upwardly at an angle with respect to the first portion 32a. The extension portion 32 may be of a rigid or flexible design and desirably is constructed from a low cost material, such as rubber, cardboard, fiberboard or plastic.

Generally, contaminants (e.g., expired particles from the patient) are difficult to re-aerosolize unless they directly contact the surface of the aerosolizing element 16 adjacent the orifices 110 (FIG. 4A). The angled second portion 32b eliminates a direct pathway from the patient back to the aerosolizing element so as to prevent expired particles (e.g., cough and sneeze particles) from directly contacting the aerosolizing element 16. Hence, this protects against patient-to-patient contamination if the aerosolizing element 16 is used to administer doses to multiple patients. The face mask 34 or other user interface can be made from a porous or non-porous material, as further described below. Other types of non-disposable or disposable patient interfaces, such as nasal prongs, oral mouthpieces, and holding chambers, also can be used with the aerosol delivery device 10.

The actuator 18 is operable to apply a moving force to the aerosolizing element 16, thereby causing the aerosolizing element to expel aerosol droplets of an agent. The actuator 18 can be any type of oscillator that can apply vibratory oscillations to the aerosolizing element 16. As best shown in FIG. 4B, the illustrated actuator 18 is a piezoelectric-driven actuator (also known as an ultrasonic horn) that includes first and second electrodes 48a, 48b, a piezoelectric element 50 disposed between the first and second electrodes, and a motion transmitting member 52 secured to the first electrode 48a. An end portion 53 of the motion transmitting member is coupled to the aerosolizing element 16.

An oscillating electric current applied to the electrodes 48a, 48b induces vibratory motion of the piezoelectric element 50, which in turn induces vibratory motion of the motion transmitting member 52 in the directions indicated by double-headed arrow 55. The motion transmitting member 52 transmits the vibratory motion to the aerosolizing element 16 for aerosolizing an agent therein. In particular embodiments, the actuator 18 generates vibrations in the range of about 20 to 200 Hz. Other types of actuators, such as a solenoid or a linear electric motor (e.g., a voice coil, such as used in a loudspeaker), also can be used to induce vibration of the aerosolizing element.

As discussed above, the actuator 18 is mounted within the air manifold 36, which directs compressed gas (e.g., compressed air) to flow over the actuator 18 and carry away heat generated during operation. The manifold 36 is formed with a flow channel 38 substantially surrounding the actuator 18 and an opening 42 that is connected to a compressed air conduit 44. The air conduit 44 receives compressed air from a compressed air source, such as the illustrated air pump 46. The manifold 36 is also formed with one or more apertures 40, which direct air in the flow channel 38 to flow through the aerosolizing element 16 in the direction of arrows 41. Air flowing through the aerosolizing element 16 entrains aerosol droplets expelled from the aerosolizing element and assists in the delivery of the droplets to the patient.

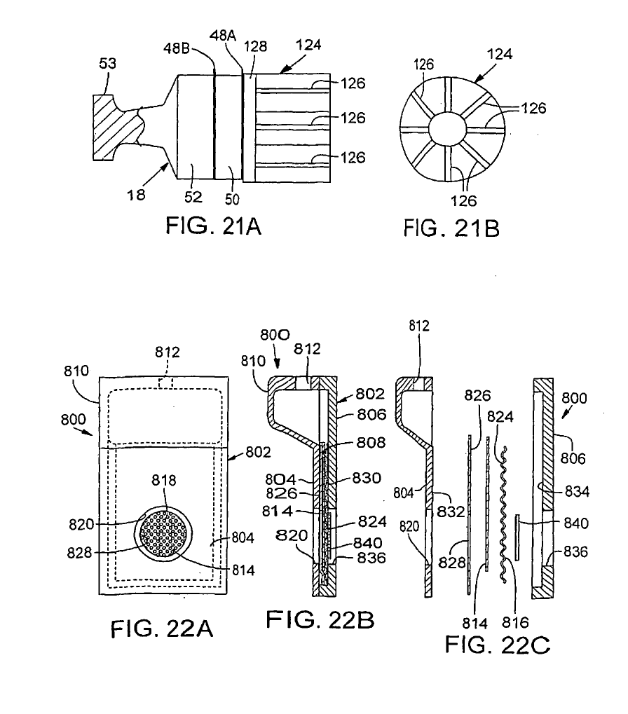

In lieu of or in addition to the air manifold 36, a heat sink 124 (FIGS. 21A and 21B) can be mounted to the actuator 18 to facilitate the dissipation of heat generated during operation. As shown in FIGS. 21A and 21B, the heat sink 124 includes a plurality of angularly spaced radial fins 126 extending longitudinally from a base 128 mounted to and in thermal contact with the actuator 18. In particular embodiments, the air manifold 36 can be sized to accommodate the actuator 18 and the heat sink 124. In other embodiments, the actuator 18 and optionally the heat sink 124 can be mounted in the housing 12 without the air manifold 36. In the latter embodiments, air from the air pump 46 can be ducted directly to the aerosolizing element 16 to assist in the delivery of aerosolized agent to the patient.

As shown in FIGS. 1A and 1B, the device 10 can include a wearable or body mountable pack or case 54 that houses the air pump 46 (e.g., a diaphragm air pump), an air filter 56, and one or more batteries 58 for powering the device. The pack 54 can be, for example, a waist pack (“fanny pack”) that can be worn around the waist of a user or a shoulder or back pack that can be worn over one or both shoulders of a user. The pack 54 also can include a controller 66, a charging jack 62 for re-charging the batteries 58, and an on/off power switch 61. The charging jack 62 can be connected to an external power supply (not shown) in a conventional manner to recharge the batteries 58 or to provide power to operate the device without use of the batteries. The pack 54 can be coupled to the housing 12 via a flexible umbilical 60 that contains the air conduit 44 and wiring connecting the controller 66 to the actuator 18 and a trigger switch 64 on the housing. By housing the pump 46, the batteries 58, and the controller 66 in the pack 54, the overall weight of the housing 12 can be reduced for easier handling. In an alternative embodiment, one or more of these components can be housed in the handle portion 14 or in another portion of the housing.

Although not shown in the illustrated embodiment, a compressed air receiver or reservoir can be housed in the handle portion 14 or the pack 54. The air reservoir can have an inlet that receives compressed air from the air pump 46 via a first conduit and an outlet that supplies a charge of compressed air to the air manifold 36 via another conduit. In another embodiment, the handle portion 14 can be equipped with a hand pump operable to charge the air receiver, such as disclosed in co-pending U.S. application Ser. No. 10/587,814 and U.S. application Ser. No. 10/471,620 (U.S. Patent Application Publication No. US-2004-0134494 issued as U.S. Pat. No. 7,225,807), which is incorporated herein by reference. The device 10 also can be equipped with a hand-crank dynamo operable to recharge the batteries 58, such as disclosed in the '814 application and the '807 patent.

The aerosol delivery device 10 can be operated in a continuous or automatic dose timing mode. A selector switch (not shown) can be provided on the handle portion 14 or on the pack 54 for manually setting the device to operate in either mode. In the continuous mode, a user depresses the trigger switch 64 on the handle portion 14, which sends a signal to the controller 66. The controller 66 sends a signal to the air pump 46 and the actuator 18 to begin operation. The aerosolizing element 16 converts an agent drawn from the vial 22 into droplets of a very small size (e.g., in a range of about 1 to 10 micrometers, although the size of the droplets can vary depending on the application). After administering a dose, the user depresses the trigger switch 64 again to turn off the actuator and the air pump.

In the automatic dose timing mode, the user first sets a timer switch (e.g., a rotary switch) (not shown) operatively connected to the controller at a desired setting corresponding to a predetermined aerosolization period (e.g., 15, 20, 30, or 60 seconds). In alternative embodiments, the device 10 can include a keypad or another type of input device to allow the user to set the desired time of application. To initiate administration of a dose, the user depresses the trigger switch 64, which activates the pump 46 to supply air to the manifold 36. After a predetermined period of time (e.g., 0.5 seconds), the actuator 18 is activated to aerosolize the agent in the aerosolizing element 16. At the end of the aerosolization period, the actuator 18 is automatically turned off, after which the aerosolization element can be purged with compressed air from the pump 46 for a predetermined period of time (e.g., 5 seconds) or until the switch 64 is depressed.

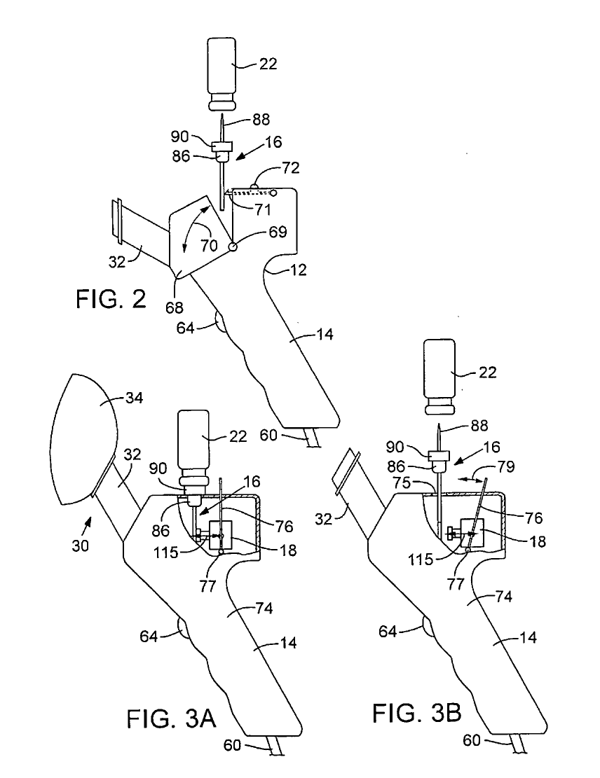

FIG. 2 shows another embodiment of the aerosol delivery device 10 that is similar to the embodiment shown in FIGS. 1A and 1B, except that it includes a hinged front portion 68 that is coupled to the housing 12 by a pivot pin 69. The front portion 68 is pivotable about the pin 69 (in the directions indicated by double-headed arrow 70) between an open position for removing or replacing the aerosolizing element 16 (as shown in FIG. 2) and a closed position in which the aerosolizing element 16 is held firmly in place between the front portion 68 and the adjacent surface of the housing 12. The housing 12 may be provided with a latch 71 that engages a corresponding surface of the front portion 68 to releasably retain the front portion 68 in the closed position. A latch button 72 on the latch 71 extends upwardly through the top of the housing 12. Depressing the latch button 72 releases the latch 71 from engagement with the front portion 68 so that it can be moved to the open position shown in FIG. 2. Various other latch or lock mechanisms can be implemented to releasably retain the front portion 68 in the closed position.

FIGS. 3A and 3B show another embodiment of the aerosol delivery device 10 that is similar in most respects to the embodiment shown in FIGS. 1A and 1B. The embodiment of FIGS. 3A and 3B includes a housing 74 formed with an upper opening 75 (FIG. 3B) that is sized to receive the aerosolizing element 16. As shown in FIG. 3A, when inserted into the opening 75, the aerosolizing element 16 is supported in an upright position by the top wall of the housing 74. The actuator 18 in this configuration is coupled to a movable lever 76 that is operable to move the actuator 18 between a first, operating position in which the actuator engages the aerosolizing element 16 (FIG. 3A) and second position in which the actuator is spaced from the aerosolizing element (FIG. 3B). The lower end of the lever 76 is pivotally mounted inside the housing 74 at a pivot pin 77 to permit pivoting of the lever in the directions indicated by double headed arrow 79. The upper end portion of the lever 76 extends through the top wall of the housing 74 for manipulation by a user. The actuator 18 is coupled to the lever 76 by a pinned connection or equivalent mechanism such that the actuator 18 is displaced along a substantially straight path (in the directions indicated by doubled-headed arrow 115) upon pivoting movement of the lever. Prior to loading the aerosolizing element 16 into the housing, the lever 76 is moved toward the rear of the housing to the position depicted in FIG. 3B. After insertion of the aerosolizing element, the lever is moved toward the front of the housing to move the actuator 18 to the operating position depicted in FIG. 3A.

Turning now to FIGS. 4A and 4B, an example of an aerosolizing element 16 will now be described. The aerosolizing element 16 has a body 78 that includes a front portion 80, a rear portion 82, a chamber 84 cooperatively formed between the front portion 80 and the rear portion 82, and an integral reservoir 86 formed at the upper end portion of the aerosolizing element and in fluid communication with the inlet of the chamber 84. A piercing prong, or needle, 88 extends upwardly from a vial mount 90 situated on top of the reservoir 86. The prong 88 has a pointed upper end that is used to puncture a puncturable septum 92 incorporated or connected to the opening of the vial 22. The septum 92 can be made of an elastomeric material (e.g., rubber) or any of various other suitable materials. The prong 88 also functions to hold the vial 22 in an inverted position on top of the vial mount 90. While the illustrated prong 88 is a small cylindrical tube, other shaped tubes, including square, triangle, or rectangle, also can be used.

The prong 88 is formed with a first flow passageway 94 extending between the upper end of the prong and the reservoir 86 to allow agent in the vial 22 to flow into the reservoir. A second flow passageway 96 in the prong 88 extends between the upper end of the prong and an air inlet, or opening, 98 formed in the vial mount 90. The opening 98 can be fitted with a porous (air permeable) plug 100 (FIG. 4B). The second flow passageway 96 allows atmospheric air to be drawn into the vial 22 to replace agent that is extracted from the vial. The reservoir 86 also can be provided with an air outlet, or opening, 102 (FIG. 4A) fitted with a porous plug (not shown) to allow for venting of air in the reservoir. The porous plugs in openings 98 and 102 are made of a material that is permeable to air but inhibits leakage of the agent due to surface tension.

The front portion 80 of the aerosolizing element 16 defines an orifice surface 104 that is formed with at least one or a plurality of orifices 110. The rear portion 82 defines a movable element 106 opposite the orifices 110 that is coupled to the end portion 53 of the actuator 18. The movable element 106 is movable or deformable to increase pressure in the chamber 84 in response to the force applied by the actuator 18. In the illustrated embodiment, for example, the movable element 106 comprises a flexible diaphragm that alternately flexes inwardly and outwardly in response to movement of the actuator. In operation, rapid motion of the actuator 18 pushes the diaphragm inwardly and toward the orifices 110, increasing pressure in the chamber 84 and expelling agent outwardly through the orifices 110 to form aerosol droplets 108. Movement of the actuator 18 in the opposite direction causes the diaphragm to flex outwardly and away from the orifices, thereby decreasing the pressure in the chamber 84 and drawing agent into the region of the chamber behind the orifices for the next cycle. In alternative embodiments, the movable portion need not be flexible or deformable but is otherwise configured to move toward and away from the front portion 80 in response to movement of the actuator 18.

As shown in FIG. 4A, the aerosolizing element 16 can be formed with one or more air flow apertures, or openings, 112 extending through a peripheral portion of the element adjacent the orifice surface 104. The openings 112 are in fluid communication with the apertures 40 of the air manifold 36 (FIG. 1B) at the rear surface of the element 16 so that air from the apertures 40 can flow through the openings 112 and entrain droplets 108 expelled by the orifices 110 for delivery to the patient.

The orifices 110 typically are about 5 micrometers in diameter, although the size of the orifices can vary depending on the desired size of the droplets 108. The front and rear portions 80, 82 can be made from any of various suitable materials, such as plastic, using conventional manufacturing techniques (e.g., molding). The orifices 110 can be formed directly in the front portion 80 using conventional micro-machining techniques, such as laser drilling, electroforming, or chemical etching. As depicted in FIG. 4B, the rear portion 82 can be of a unitary construction having a substantially constant thickness. In other embodiments, the rear portion can have a relatively thinner section opposite the orifices 110 that defines the movable element 106. In another embodiment (e.g., the aerosolizing element 800 shown in FIGS. 22A-22C, which is described below), the movable element can be a separate element bounding the chamber opposite the orifices. In the latter embodiment, the rear portion 82 can be formed with an opening to receive the actuator 18 for coupling to the movable element.

Preferably, the aerosolizing element 16 is disposable. If the device is used where disposal costs are not prohibitive (e.g., in a modern hospital), the aerosolizing element (and the mask 34 or other user interface) can be disposed of each time a dose is administered to a patient. However, if the device is used in a high workload application, such as a mass vaccination campaign, disposal costs may be a concern. In such cases, the aerosolizing element can be used to administer doses to multiple patients, but typically would be disposed of after a session of administering multiple doses to prevent the growth of bacteria or other contaminants. Notably, the aerosolizing element 16 inhibits contact of the agent with the actuator 18 and other re-useable components of the device 10. Consequently, substantially less time is required for cleaning and maintenance of the device compared to conventional nebulizers.

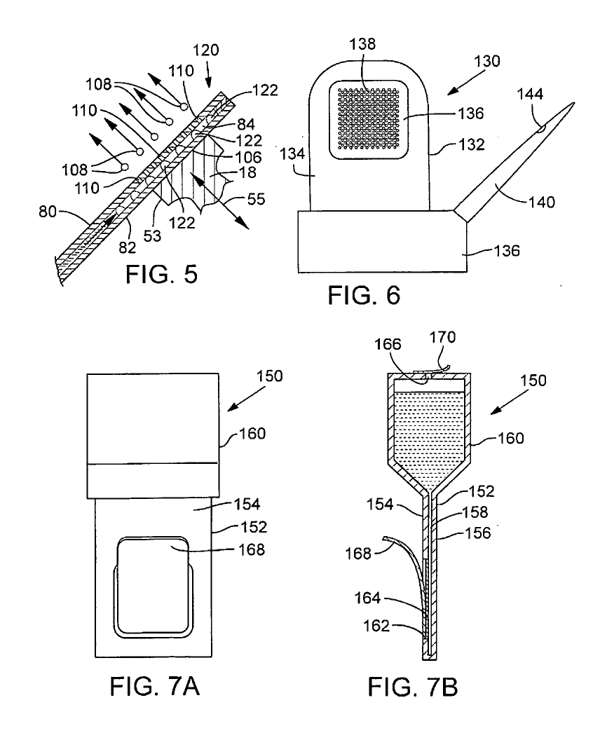

FIG. 5 shows another example aerosolizing element 120, according to another embodiment, that can be used in any of the aerosol delivery devices described herein. The aerosolizing element 120 is similar to the aerosolizing element 16, except that agent is drawn upwardly to the area of the chamber 84 behind the orifices 110 by capillary action. The aerosolizing element 120 can be provided with a piercing prong 88 for drawing agent upwardly from a vial 22 (not shown in FIG. 5). Alternatively, rather than drawing agent from a vial, the aerosolizing element 120 can include an integral reservoir sized to receive a predetermined quantity of an agent sufficient for supplying a single dose or multiple doses.

The thickness of the chamber 84 (the distance measured between the opposed internal surfaces of the front and rear portions 80, 82) is selected to maintain an adequate flow of agent via capillary action without inducing a pressure loss that exceeds the capillary head. As shown, the aerosolizing element 120 can include one or more spaced apart dimples, or projections, 122 disposed in the chamber 84. The projections 122 maintain a minimum spacing in the chamber 84 between the movable portion 106 and the front portion 80 of the element so as to maintain adequate capillary head without undue pressure loss.

FIG. 6 shows an aerosolizing element 130, according to another embodiment, that can be used in any of the aerosol delivery devices described herein. The aerosolizing element 130 has a body 132 that includes a first portion 134 and a second, deformable portion 136 that serves as a reservoir for an agent to be aerosolized. The first portion 134 has a construction that is similar to the aerosolizing element 16 in that it includes an internal chamber (not shown) for receiving an agent to be aerosolized, an orifice area 136 defining a plurality of orifices 138, and a movable portion (not shown) bounding the chamber opposite the orifices 138 for forcing agent through the orifices 138.

The deformable portion 136 of the aerosolizing element 130 is made of a flexible, resilient material, such as rubber or another suitable elastomer. A piercing prong 140 extends from the deformable portion 136 for insertion into a vial 22. The piercing prong 140 is formed with an opening 144 to receive agent from the vial. The deformable portion 136 functions in a manner similar to the squeeze bulb on a conventional eyedropper.

Prior to inserting the prong 140 into a vial, the user squeezes the deformable portion 136. After insertion, finger pressure is removed from the deformable portion 136, allowing it to return to its normal shape and thereby drawing agent from the vial via the prong 140. The agent in the deformable portion 136 can be fed into the chamber of the first portion 134 via gravity or capillary action, as described above in connection with the embodiments shown in FIGS. 4A, 4B, and 5.

FIGS. 7A and 7B show an aerosolizing element 150, according to another embodiment, that can be used in any of the aerosol delivery devices described herein. The aerosolizing element 150 has a body 152 that includes a front portion 154, a rear portion 156, a chamber 158 cooperatively formed between the front portion 154 and the rear portion 156, and an enlarged reservoir 160 formed at the upper end portion of the element and in fluid communication with the inlet of the chamber 158.

The reservoir 160 can be sized to hold a predetermined volume of agent sufficient to deliver a single dose or multiple doses. The reservoir 160 desirably is provided with a venting port 166 to expose the interior of the reservoir to atmosphere when agent is drawn from the reservoir into the chamber 158. Although not shown in FIGS. 7A and 7B, a removable piercing prong can be inserted into the venting port 166 for supplying agent to the reservoir 160 from a vial 22. Additionally, the reservoir 60 can be filled via port 166 using a needle and syringe or equivalent device. The agent can be fed from the reservoir 160 into the chamber 158 via gravity or capillary action.

The front portion 154 is formed with an opening 162 (FIG. 7B) in which there is fitted an orifice plate 164 having multiple orifices for expelling droplets of agent. In one implementation, the aerosolizing element 150 is filled with a predetermined volume of agent and sealed by a pharmaceutical manufacturer or pharmacy. In this regard, a removable sealing tape 168 or other similar type adhesive or layer can be placed over the orifice plate 164 to prevent leakage of agent and the ingress of foreign matter and other desired material into element prior to use. Likewise, a removable sealing tape 170, a removable tab or other closure can be used to close the venting port 166. The sealing tapes 168 and 170 are then removed by the user prior to administering the agent.

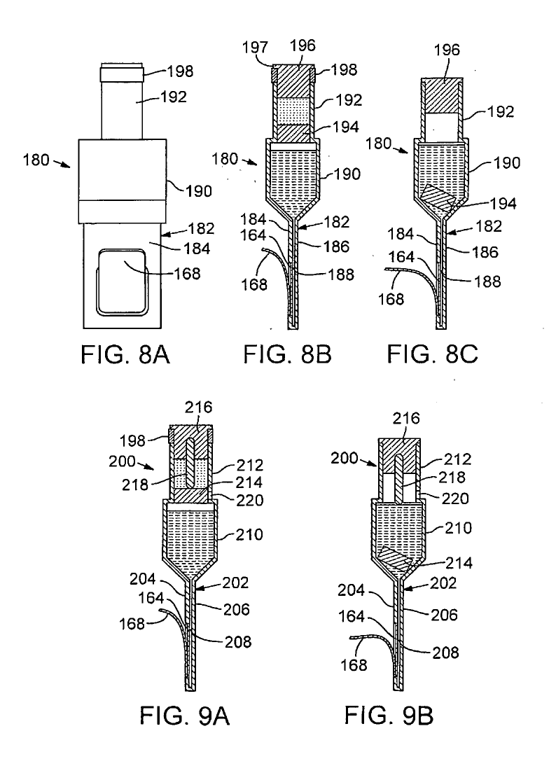

FIGS. 8A-8C show an aerosolizing element 180, according to yet another embodiment, that can be used in any of the aerosol delivery devices described herein. The aerosolizing element 180 differs from the previously described embodiments in that it can be used to store and mix two different liquid components. As shown, the aerosolizing element 180 has a body 182 that includes a front portion 184, a rear portion 186, a chamber 188 cooperatively formed between the front portion 184 and the rear portion 186, a first reservoir 190 in fluid communication with the inlet of the chamber 188, and a second reservoir 192 defined at the upper end portion of the aerosolizing element. An orifice plate 164 is disposed in an opening formed in the front portion 184.

As shown in FIG. 8B, the chamber 188 and the first reservoir 190 are filled with a first liquid and the second reservoir 192 is filled with a second liquid. A plug, or separation element, 194 is disposed in the aerosolizing element 180 between the first and second reservoirs to keep the liquids separated from each other prior to use. The second reservoir 192 has an open top that is fitted with a plug 196. A removable, annular ring 198 is disposed around the plug 196 and seated against the open end of the second reservoir 192. The plug 196 is formed with an annular flange portion 197 that overlaps the ring 198. The ring 198 prevents inadvertent or premature mixing of the first and second liquids by resisting movement of the plug 196 into the second reservoir 192.

To reconstitute the first and second liquids at the time of use, the user removes the ring 198 and pushes down on the plug 196 to pressurize the second reservoir 192. Due to the incompressibility of the liquid, the liquid forces the plug 194 into the wider area of the first reservoir 190, thereby allowing the liquid in the second reservoir to mix with the liquid in the first reservoir (as shown in FIG. 8C). In use, the agent can be fed from the reservoir 190 into the chamber 188 via gravity or capillary action.

FIGS. 9A and 9B show an aerosolizing element 200, according to yet another embodiment, that can be used in any of the aerosol delivery devices described herein. The aerosolizing element 200 differs from the previously described embodiments in that it can be used to store and mix a liquid component and a dry component (e.g., a solid or powdered component). As shown, the aerosolizing element 200 has a body 202 that includes a front portion 204, a rear portion 206, a chamber 208 cooperatively formed between the front portion 204 and the rear portion 206, a first reservoir 210 in fluid communication with the inlet of the chamber 208, and a second reservoir 212 defined at the upper end portion of the aerosolizing element. An orifice plate 164 is disposed in an opening formed in the front portion 204.

As shown in FIG. 9A, the chamber 208 and the first reservoir 210 are filled with a liquid (e.g., a diluent for a dry component) and the second reservoir 212 is filled with a powder (e.g., lyophilate) or another type of dry component. A plug 214 is disposed in the aerosolizing element 200 between the first and second reservoirs to keep the dry component separated from the liquid component prior to use. The second reservoir 212 has an open top that is fitted with a plug 216. A rigid push rod 218 (e.g., a glass rod) extends from the plug 216 and contacts the plug 214 (FIG. 9A). The body 202 can be formed with a venting port 220 between the first and second reservoirs 210 and 212 adjacent the plug 214. As shown in FIG. 9A, the plug 214 covers the port 220 to prevent leakage prior to use.

To reconstitute the liquid and dry components at the time of use, the user removes the ring 198 and pushes down on the plug 216. Movement of the plug 216 and the push rod 218 forces the plug 214 into the wider area of the first reservoir 210, thereby allowing the dry component in the second reservoir to mix with the liquid in the first reservoir and form an agent for administering to a patient (as shown in FIG. 9B). Displacement of the plug 214 also exposes the first reservoir 210 to atmospheric pressure via the venting port 220 to facilitate the flow of agent into the chamber 208. In use, the agent can be fed from the reservoir 210 into the chamber 208 via gravity or capillary action.

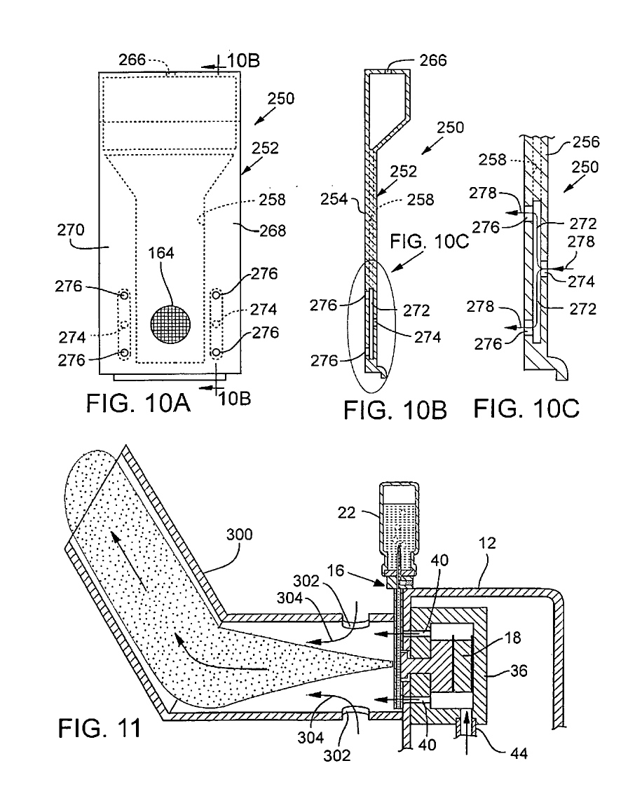

FIGS. 10A-10C show another example aerosolizing element 250, according to another embodiment, that can be used in any of the aerosol delivery devices described herein. The aerosolizing element 250 has a body 252 that includes a front portion 254, a rear portion 256, a chamber 258 cooperatively formed between the front portion 254 and the rear portion 256, and an integral reservoir 260 formed at the upper end portion of the aerosolizing element and in fluid communication with the inlet of the chamber 258. The reservoir 260 desirably is provided with a venting port 266 to expose the interior of the reservoir to atmosphere pressure when agent is drawn from the reservoir into the chamber 258. In use, the agent can be fed from the reservoir 260 into the chamber 258 via gravity or capillary action.

The front portion 254 is formed with an opening in which there is fitted an orifice plate 164 for expelling droplets of agent. The body 252 further includes peripheral portions 268, 270 on opposite sides of the chamber 258 (FIG. 10A). Formed in the peripheral portions 268, 270 are respective air flow passageways 272 (FIGS. 10B and 10C). As best shown in FIG. 10C, each passageway 272 extends from an inlet 274 formed in the rear portion 256 to one or more outlets 276 formed in the front portion 254 at locations offset from the inlet 274. When the aerosolizing element 250 is placed in the housing of an aerosol delivery device (e.g., the device 10 shown in FIGS. 1A and 1B), the inlets 274 are positioned to receive compressed air from the air manifold 36. Air flows into the inlets 274, through the passageways 272 and exits the outlets 276 (as indicated by arrows 278) to entrain droplets expelled by the orifice plate 164. Because the outlets 276 are offset from the inlet 274, there is less likelihood that expired particles from the patient can travel through the passageways and contact the actuator 18 or other reusable portions of the system.

FIGS. 22A-22C show another example aerosolizing element 800, according to another embodiment, that can be used in any of the aerosol delivery devices described herein. The aerosolizing element 800 has a body 802 that includes a front portion 804, a rear portion 806, and a reservoir 810 formed at the upper end portion of the aerosolizing element. The reservoir 810 desirably is provided with a venting port 812.

Disposed between the front and rear portions 804, 806 is an orifice plate 814 (e.g., an electroformed mesh plate) and a flexible spacer element 816. A chamber 808 for receiving agent from the reservoir 810 is defined between the orifice plate 814 and the spacer element 816. The orifice plate 814 is formed with a plurality of orifices 818 that are aligned with an opening 820 in the front portion 804. The spacer element 816 is formed with a plurality of projections 824 that maintain a minimum spacing in the chamber 808 between the orifice plate 814 and the spacer element 816. Although not required, the orifice plate 814 and the spacer element 816 can be held together by a piece of adhesive tape 826 placed over the orifice plate and secured to the lower end portion of the spacer element for ease of assembly. The tape 826 is formed with an opening 828 aligned with the opening 820 in the front portion 804. The rear portion 806 is formed with an opening 836 that is sized to receive the front end portion 53 of the actuator 18 (FIG. 4B). A piece of double-sided tape 840 can be used to secure the end portion 53 of the actuator 18 to the spacer element 816. A suitable sealant (e.g., silicone) can be used to secure the tape 826 to the inside surface 832 of the front portion 804 and to secure the spacer element 816 to the inside surface 834 of the rear portion 806.

In particular embodiments, the orifice plate 814 comprises a thin metal foil (e.g., nickel, aluminum, gold, or another suitable metal) having a thickness of about 0.05 mm. Other suitable materials, such as ceramics or composite materials, including but not limited to plastics, also can be used to form the orifice plate 814. The orifices 818 can be formed using conventional micro-machining techniques, such as laser drilling, electroforming, and chemical etching. The spacer element 816 comprises a thin flexible plastic having a thickness of about 0.1 mm. The projections 824 on the spacer element 818 have a height of about 0.1 mm. Of course, these specific dimensions (as well as other dimensions provided in the present specification) and materials are given to illustrate the invention and not to limit it. The dimensions and materials provided herein can be modified as needed in different applications or situations.

The spacer element 816 serves as a flexible diaphragm for expelling agent through the orifice plate 814. In use, the end portion 53 of the actuator 53 extends through the opening 836 and bears against the spacer element 816. Vibration of the actuator 18 is transmitted to the spacer element 816, causing it to flex toward and away from the orifice plate 814, alternately forcing agent in the chamber 808 through the orifices 818 and drawing agent into the chamber 808 from the reservoir 810.

FIG. 11 shows an extension portion 300 of a patient interface that can be used with the aerosol delivery device 10 (or other delivery devices), according to another embodiment. The extension portion 300 is similar to the extension portion 32 shown in FIGS. 1A and 1B, except that the extension portion 300 includes one or more openings, or vents, 302 proximate the housing 12. A user interface 34 (not shown in FIG. 11) such as a mask or other appropriate user interface, such as nasal prongs or mouth pieces can be coupled to the end of the extension portion 300 in the manner shown in FIGS. 1A and 1B. The openings 302 allow inspiratory air to be drawn into the extension portion 300, as indicated by arrows 304, so as to allow the patient to breathe normally during the administration of an agent. As outside air enters the extension portion, the air entrains aerosol droplets expelled by the aerosolizing element 16 to assist in the delivery of droplets to the patient.

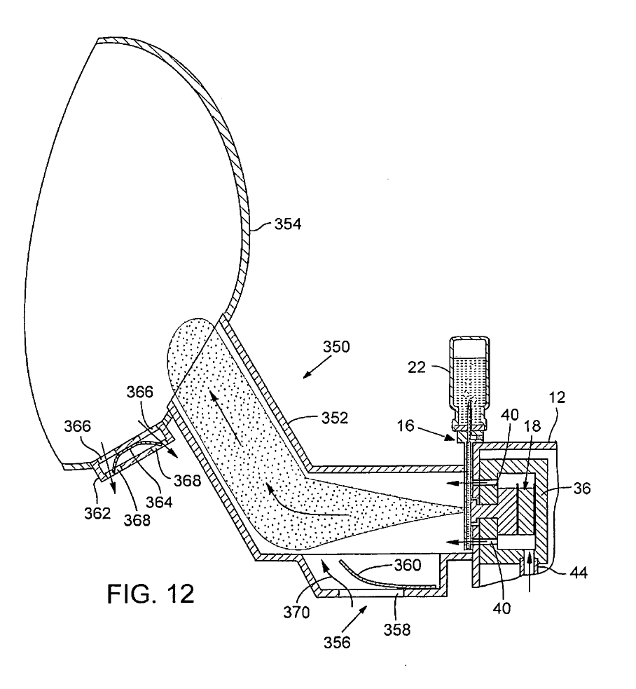

FIG. 12 shows a patient interface 350 that can be used with the aerosol delivery device 10 (or other delivery devices), according to another embodiment. The patient interface 350 includes an extension portion 352 extending from the housing 12 and a mask 354 or other user interface coupled to the end of the extension portion 352. The extension portion 352 includes a one-way valve 356 that is operable to allow inspiratory air to flow into the extension portion and inhibit flow in the opposite direction to the surrounding environment. The illustrated valve 356 includes an opening 358 formed in the extension portion 352 and a flexible sealing member 360 secured at one end to the inside surface of the extension portion. The sealing member 360 can be made from a flexible and/or elastomeric material, such as rubber or any of various other suitable elastomers. In its normal, at rest position, the sealing member 360 covers the opening 358. During inhalation, the sealing member 360 opens to allow outside air to be drawn into the extension portion through the opening 358 (as indicated by arrow 370) to assist in the delivery of aerosol droplets to the patient. During exhalation, the sealing member 360 covers the opening 358 to prevent aerosolized agent in the extension portion from being released to the surrounding environment.

The mask 354 or other user interface in this embodiment is made of a non-porous material (a material that does not allow passage of air) and includes a one-way valve 362 to allow for the release of expiratory flow. The valve 362 houses a flexible sealing member 364 that covers openings 366 in the mask or other user interface in its normal, at rest position to prevent outside air from flowing into the mask or other user interface. During exhalation, the sealing member 364 opens to allow expiratory air to flow through openings 366 and openings 368 to the environment.

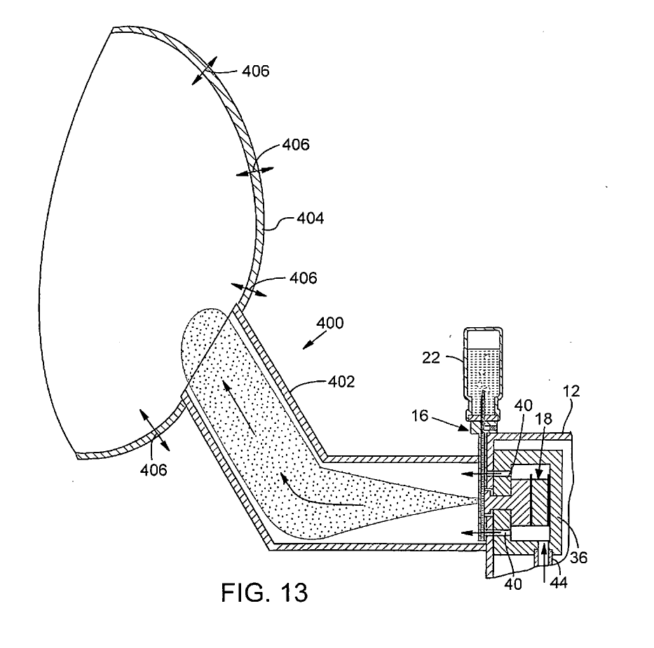

FIG. 13 shows a patient interface 400 that can be used with the aerosol delivery device 10 (or other delivery devices), according to another embodiment. The patient interface 400 includes an extension portion 402 extending from the housing 12 and a mask 404 or other user interface coupled to the end of the extension portion 402. The mask 404 or other user interface in this embodiment is made of a porous material that allows for the passage of air. In one example, the mask 404 can be manufactured from, for example, non-woven polypropylene, such as used in conventional surgical or dust masks, or other suitable materials. Expiratory and inspiratory air can flow through the mask 404 or other user interface (as indicated by double-headed arrows 406), but traps expired particulates (e.g., cough and sneeze particles) and aerosolized agent in the mask or other user interface from being released to the environment. The extension portion 402 can include a one-way valve 356 (FIG. 12) to permit outside air be drawn into the flow path and assist in the delivery of aerosol droplets expelled by the aerosolizing element 16.

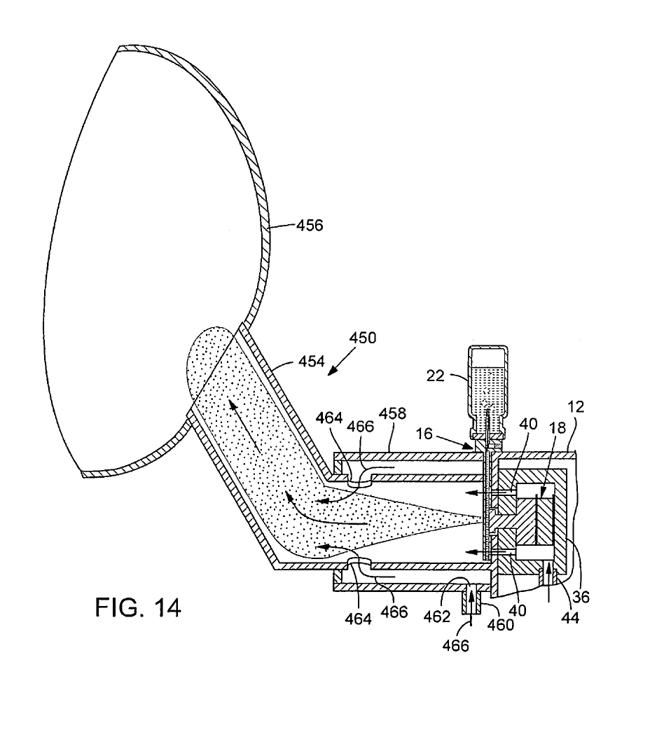

FIG. 14 shows a patient interface 450 that can be used with the aerosol delivery device 10 (or other delivery devices), according to another embodiment. The patient interface 450 includes an extension portion 454 extending from the housing 12, a mask 456 or other user interface coupled to the end of the extension portion 454, and an air distribution plenum 458 co-axially disposed around the horizontal portion of the extension portion 454. A compressed air conduit 460 is connected to an air inlet 462 of the plenum 458 to deliver compressed air from the pump 46 (FIGS. 1A and 1B) (or another source of compressed air) to the plenum 458. The extension portion 454 is formed with one or more openings 464 inside of the plenum 458. In use, compressed air from the conduit 460 flows into the plenum 458, though openings 464 and into the extension portion 454 (in the direction of arrows 466). The air flow from the plenum further assists in the delivery of the aerosol droplets to the patient and reduces aerosol deposition on the internal surfaces of the extension portion by directing the aerosol droplets away from these surfaces.

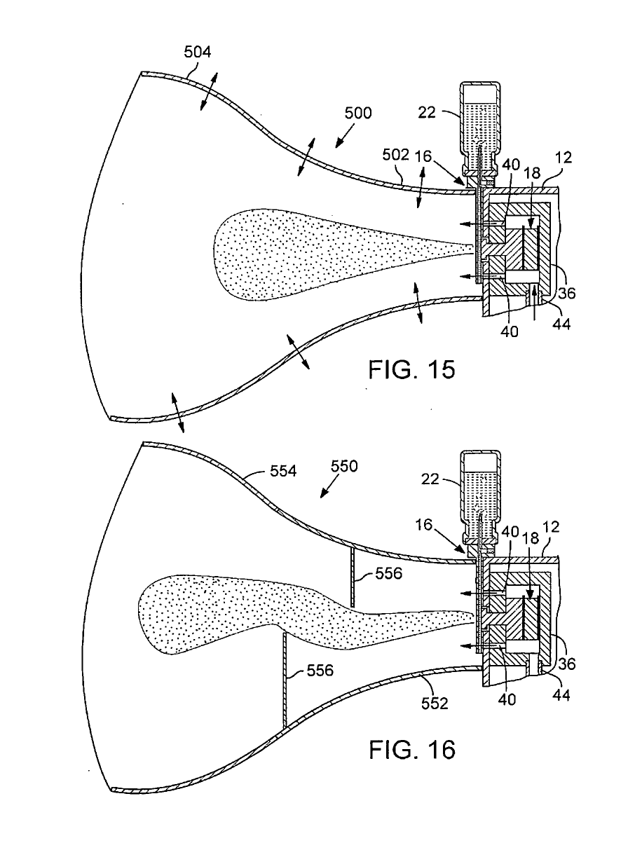

FIG. 15 shows a patient interface 500 that can be used with the aerosol delivery device 10 (or other delivery devices), according to another embodiment. The patient interface 500 includes a first portion 502 extending from the housing 12 and a second, enlarged portion 504 sized to cover the nose and mouth of a patient. The patient interface 500 is made of a porous material to allow for the passage of expiratory and inspiratory air along the entire length of the interface. In the particular embodiments, the entire patient interface 500 is intended to be disposed of after each use to protect against patient-to-patient, or alternatively a use-by-sue (e.g. in cases one user or self-infective) contamination.

FIG. 16 shows a patient interface 550 that can be used with the aerosol delivery device 10 (or other delivery devices), according to another embodiment. The patient interface 550 includes a first portion 552 extending from the housing 12 and a second, enlarged portion 554 sized to cover the nose and mouth of a patient. Depending on the embodiment, at least one or a plurality of baffles 556 are spaced along the length of the first portion 552 and extend into the flow path of aerosol droplets expelled from the aerosolizing element 16. The baffles 556 shield the aerosolizing element 16 and other re-usable components from expired particles (e.g., cough or sneeze particles) to protect against patient-to-patient contamination. In the illustrated embodiment, the first portion 552 is made of a non-porous material and the second portion 554 is made of a porous material. The first and second portions 552, 554 can be secured to each other using suitable techniques or mechanisms, such as adhesives or fasteners. Alternatively, the entire patient interface 550 can be made from single piece of porous material, similar to the patient interface 500 of FIG. 15, or from two separately formed pieces of porous material that are joined together to form the patient interface. The patient interface 550, like the patient interface 500, preferably is detachable either as a re-usable or a disposable.

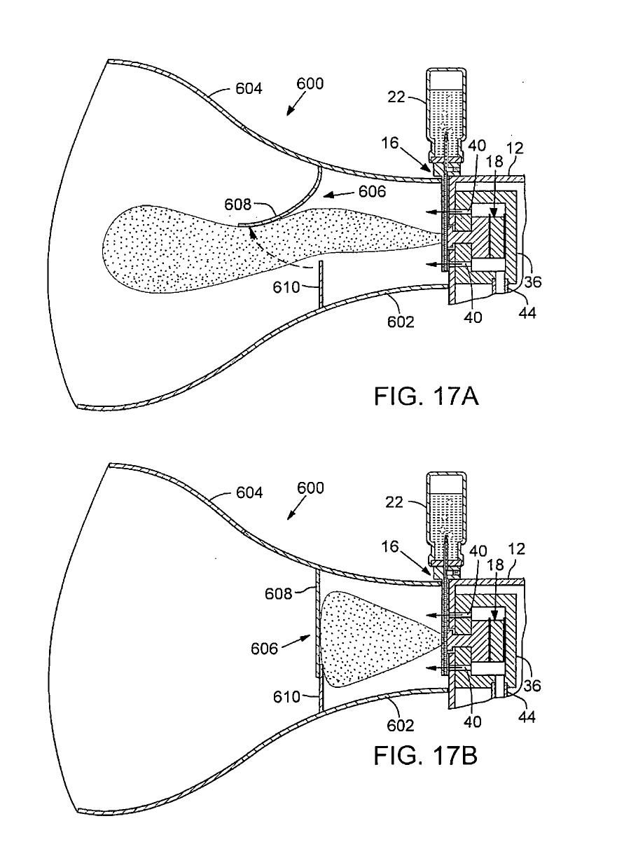

FIGS. 17A and 17B shows a patient interface 600, according to another embodiment, that includes a first portion 602 extending from the housing 12 and a second, enlarged portion 604 sized to cover the nose and mouth of a patient. The second portion 604 is made of a porous material while the first portion 602 may be made of a porous or non-porous material. The patient interface 600 is similar to the patient interface 550 of FIG. 16, except that the patient interface 600 includes a one-way valve 606 disposed in the first portion 602. The valve 606 is a flapper-type valve having a flexible sealing member 608 secured at one end to the inside surface of the first portion 602 and a non-movable valve seat 610 secured at one end to the inside surface of the first portion 602 opposite the sealing member 608.

In its normal, at rest position, the sealing member 608 contacts or partially overlaps the valve seat 610 to close the flow path from the aerosolizing element 16 to the patient (FIG. 17B). During inhalation, the sealing member 608 opens to allow aerosol droplets and air to flow to the patient (FIG. 17A). During exhalation, the valve closes (FIG. 17B) to protect the aerosolizing element 16 and other re-useable components against contamination from expired particles. In another embodiment, the patient interface 600 can include both the valve 606 and baffles 566 (FIG. 16) to further protect against contamination. The patient interface 600, like the patient interface 500, preferably is disposable.

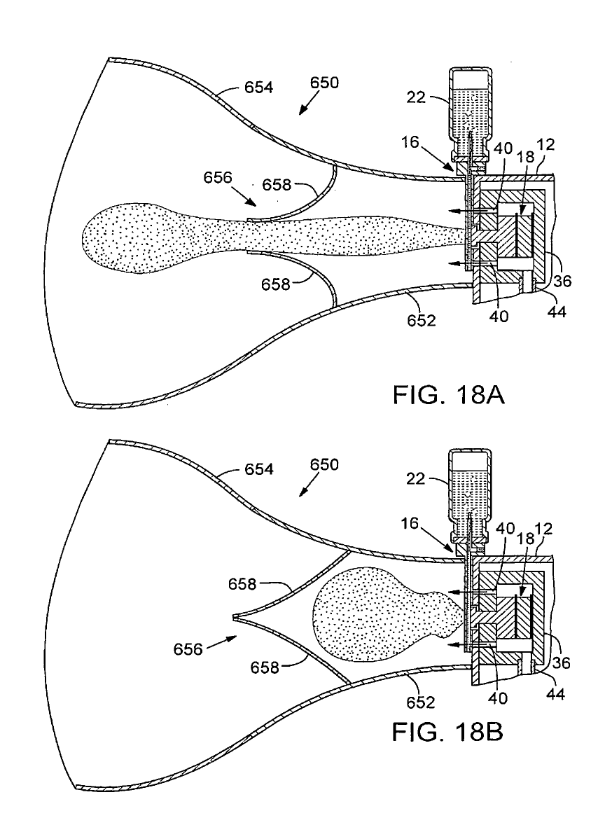

FIGS. 18A and 18B shows a patient interface 650, according to another embodiment, that includes a first portion 652 extending from the housing 12 and a second, enlarged portion 654 sized to cover the nose and mouth of a patient. The second portion 654 is made of a porous material while the first portion 652 may be made of a porous or non-porous material. The patient interface 650 is similar to the patient interface 600 of FIGS. 17A and 17B, except that the patient interface 650 includes a one-way, “duckbill” type valve 656 disposed in the first portion 652. The valve 656 includes first and second flexible sealing members 658, each of which is connected to the inside surface of the first portion 652. The sealing members 658 extend toward and contact each at their free ends so as to close the flow path from the aerosolizing element 16 to the patient when the valve is in its normal, at rest position (FIG. 18B). The sealing members 658 may be made of any of various suitable elastomeric materials. During inhalation, the sealing member 658 open to allow aerosol droplets and air to flow to the patient (FIG. 18A). During exhalation, the valve closes (FIG. 18B) to protect the aerosolizing element 16 and other re-useable components against contamination from expired particles. The patient interface 650, like the patient interface 500, preferably is disposable.

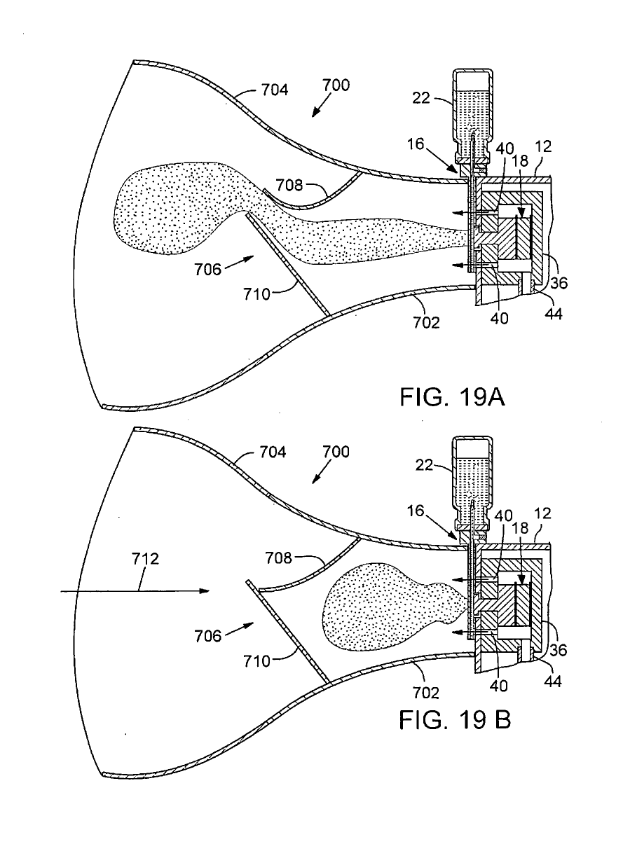

FIGS. 19A and 19B shows a patient interface 700, according to another embodiment, that includes a first portion 702 extending from the housing 12 and a second, enlarged portion 704 sized to cover the nose and mouth of a patient. The second portion 704 is made of a porous material while the first portion 702 may be made of a porous or non-porous material. The patient interface 700 includes a one-way flapper-type valve 706 that includes a flexible sealing member 708 secured at one end to the inside surface of the first portion 702. A generally rigid seating member 710 is secured to the first portion 702 opposite the flexible sealing member 708. The seating member 710 is angled away from the housing 12 and extends to a location at or above the longitudinal center of the patient interface 700 so as to shield the aerosolizing element 16 from expired particles. The valve 706 operates in similar manner to the valve 606 shown in FIGS. 17A and 17B to allow flow from the aerosolizing element 16 to the patient and restrict flow in the opposite direction during exhalation. The patient interface 700, like the patient interface 500, preferably is disposable.

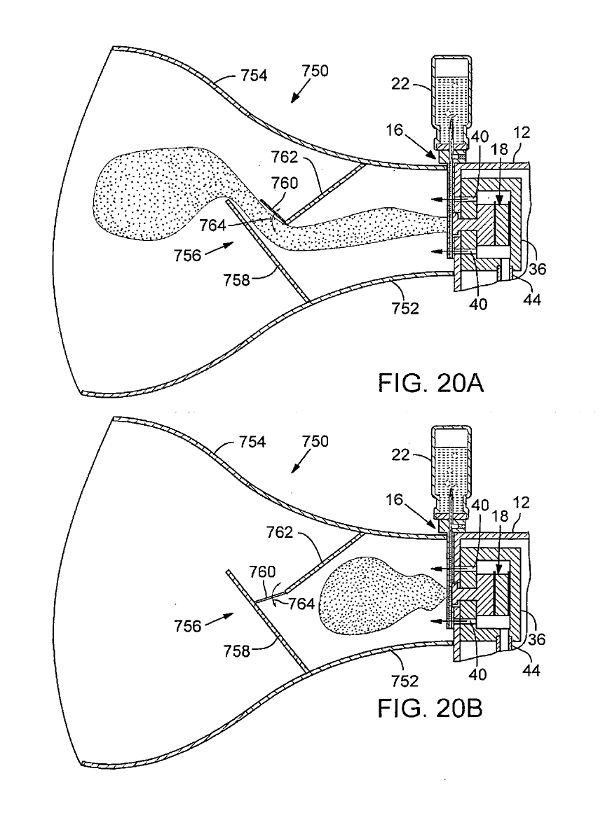

FIGS. 20A and 20B shows a patient interface 750, according to another embodiment, that includes a first portion 752 extending from the housing 12 and a second, enlarged portion 754 sized to cover the nose and mouth of a patient. The second portion 754 is made of a porous material while the first portion 752 may be made of a porous or non-porous material. The patient interface 750 includes a one-way valve 756 that includes a generally rigid seating member 758 secured to the first portion 752. A hinge assembly includes a support plate 762 secured to the first portion opposite the seating member 758 and a sealing member 760 pivotally connected to the support plate 762 for pivoting movement in the directions indicated by double-headed arrow 764. In its normal at rest position, the sealing member 760 rests against the seating member 758 (FIG. 20B) to close the valve. During inhalation, the sealing member 760 pivots upwardly and away from the seating member 758 to allow aerosol droplets and air to flow to the patient (FIG. 20A). During exhalation, the sealing member 760 returns to the closed position to restrict flow in the opposite direction (FIG. 20B).

Although the patient interfaces shown in FIGS. 11-20 are shown being used in an aerosol delivery device having an actuator 18 and an aerosolizing element 16, this is not a requirement. Accordingly, the patient interfaces can be implemented in other types of aerosol delivery systems, including without limitation, ultrasonic, vibrating mesh, jet nebulizer systems and pneumatic aerosol delivery systems.

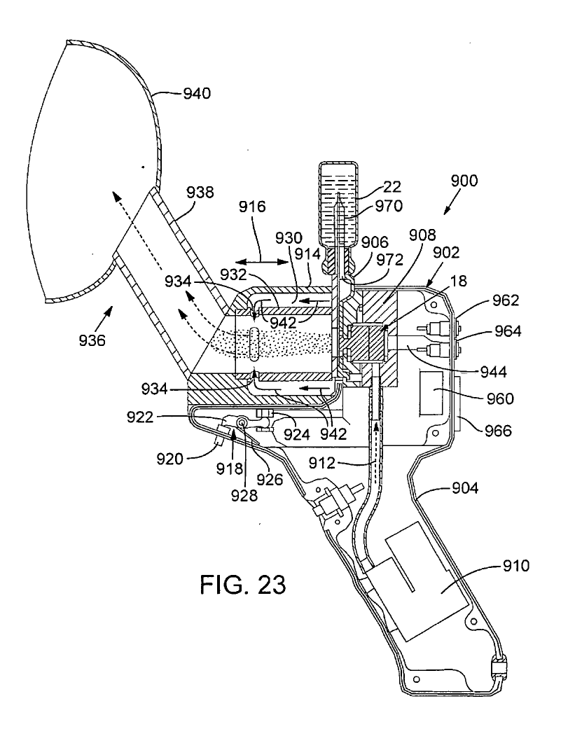

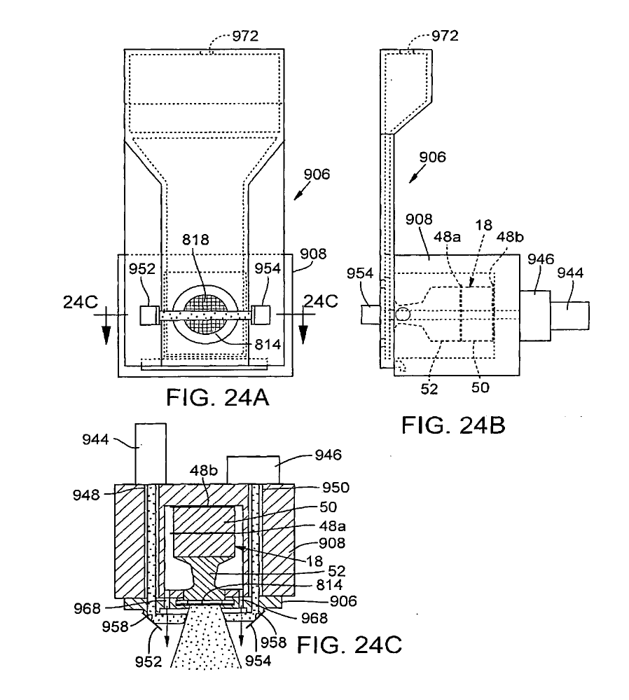

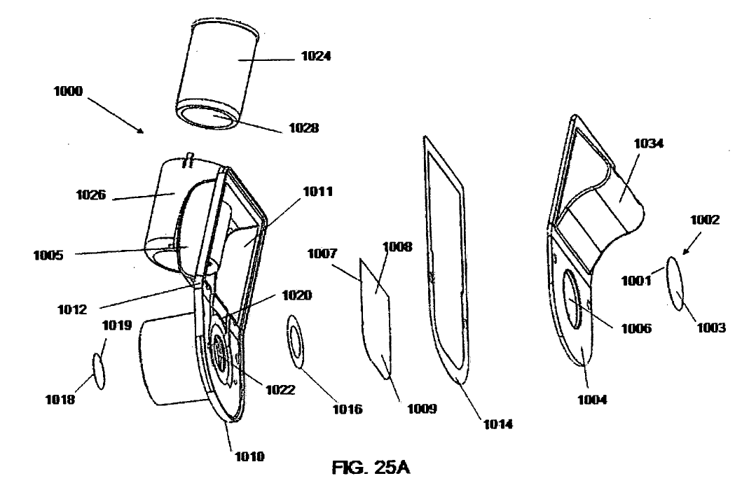

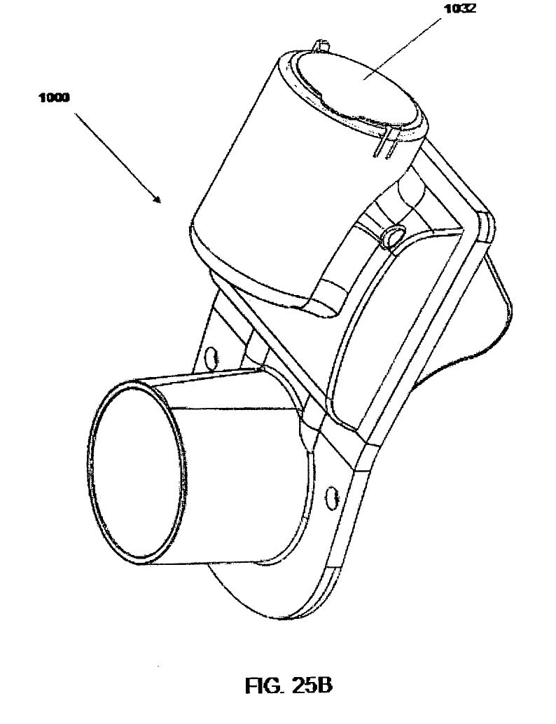

FIGS. 23 and 24A-24C show an aerosol delivery device 900, according to another embodiment. The aerosol delivery device 900 includes a body, or housing, 902 formed with a handle portion 904 shaped to be held in a user's hand. The housing 902 houses a removable aerosolizing element 906, an actuator 18, and an air manifold 908 substantially surrounding the actuator 18. The aerosolizing element 906 has a construction that is similar to the construction of the aerosolizing element 800 shown in FIGS. 22A-22C. Thus, components in FIGS. 23 and 24A-24C that are similar to components in FIGS. 22A-22C are given the same reference numerals and are not described further. As shown in FIG. 23, the aerosolizing device 906 further includes a piercing prong 970 extending from a venting port 972 into a vial 22.

The handle portion 904 houses an air pump 910 that is fluidly coupled to the air manifold 908 via an air conduit 912. A first indicator light 962 on the housing 902 provides a visual indication of whether an agent is being aerosolized. A second indicator light 964 provides a visual indication of whether the aerosolization rate is outside a predetermined, acceptable range. The indicator lights 962, 964 can be, for example, LEDs or lamps.

A front portion 914 of the housing 902 is mounted for sliding movement toward and away from the aerosolizing element 906, as indicated by double-headed arrow 916. In its closed, operating position (as shown in FIG. 23), the front portion 914 holds the aerosolizing element 906 firmly in place against the actuator 18. The front portion 914 can be moved to an open position spaced from the housing 902 to access the aerosolizing element 906.

A latch mechanism 918 for releasably retaining the front portion 914 in the closed position comprises a button 920 extending through the housing, a lever 922 connected to the housing by a pivot pin 928, and a latch pin 924 extending upwardly into a corresponding latch opening in the front portion 914. One end the lever 922 is coupled to the latch pin 924 and the opposite end of the lever bears against the button 920. A torsion spring 926 disposed around the pivot pin 928 biases the lever 922 in the counterclockwise direction in FIG. 23 to retain the latch pin 924 in the latch opening in the front portion 914. Depressing the button 920 moves in the lever 922 in the clockwise direction, this, in turn, removes the latch pin 924 from the latch opening so that the front portion 914 can be moved to the open position. The front portion 914 desirably is completely removable from the housing 902 for ease of cleaning.

The front portion 914 defines an air flow plenum 930 in fluid communication with the manifold 908 and a co-axially extending inner conduit 932 that receives aerosolized agent from the aerosolizing element 906. The inner conduit 932 is formed with one or more openings 934 in fluid communication with the air flow plenum 930. Coupled to the front portion 914 is a patient interface 936 that includes an upwardly angled extension portion 938 and a face mask 940. The extension portion 938 desirably is connected to the forward portion 914 in a removable manner for ease of cleaning or for disposal.

In use, air from the air pump 910 flows into the manifold 908 via the conduit 912 to cool the actuator 18. A portion of the airflow is ducted into the internal conduit 932 via openings 98 in the aerosolizing element 906 (FIG. 24C) to assist in carrying aerosol droplets to the patient. Another portion of the airflow in the manifold 908 is ducted into the air flow plenum 930 and then into the inner conduit 932 via openings 934, as indicated by arrows 942. The airflow from the plenum 930 assists in preventing deposition of aerosol droplets on the inner conduit 932 by directing the flow of aerosol droplets away from the inner surface.

The aerosol delivery device 900 also includes an aerosolization rate monitor that is operable to monitor the rate at which an agent is being aerosolized by the aerosolizing element 906 by detecting the obscuration of a light beam passing through an aerosol plume emanating from the aerosolization element 906. Referring also to FIGS. 24A-24C, the aerosolization rate monitor includes a light source 944 (e.g., a diode laser or a light emitting diode (LED)) and a light detector, or sensor, 946 (e.g., a photodiode), both of which are coupled to the rear surface of the manifold 908. First and second passageways 948 and 950, respectively, extend between the front and rear surfaces of the manifold 908. The aerosolization element 906 includes first and second reflectors 952 and 954, respectively, positioned on opposite sides of an orifice plate 814. Each reflector 952, 954 has a reflective surface 958 positioned at approximately a 45 degree angle with respect to the first and second passageways 948, 950 in the manifold 908.

The light source 944 projects a light beam through the first passageway 948, the aerosolization element 906, and onto the reflective surface 958 of the first reflector 952. The first reflector 952 reflects the light beam across the aerosol plume emanating from the aerosolization element 906 and onto the reflective surface 958 of the second reflector 954. The second reflector 954 reflects the light beam back through the aerosolization element 906 and the second passageway 950 toward the light detector 946. The aerosolization element 906 desirably is made of a transparent material (e.g., clear plastic) to transmit the incident and reflected light beam. Alternatively, the aerosolization element 906 can be made of a non-transparent material having openings aligned with the first and second passageways 948, 950 to allow the incident and reflected light beam to pass through the aerosolization element. The reflective surfaces 958 can be formed by applying reflective paint or a layer of reflective material (e.g., reflective tape) on the reflectors 952, 954.

As the aerosol plume passes through the reflected light beam (as best shown in FIG. 24C), the light detector 946 detects the obscuration of the light beam, which corresponds to the concentration of aerosol droplets in the aerosol plume. The light detector 946 relays a signal to a controller 960 (FIG. 23), which determines the aerosolization rate. If the aersolization rate is outside of the acceptable range, the indicator light 964 illuminates or begins flashing to provide a visual indication of this condition. The system 900 also can include a digital readout 966 (FIG. 23) mounted at a convenient location on the housing 902 to provide a digital readout of the aerosolization rate. Other indicating devices, such as an audible alarm, also can be used to provide the user information regarding the operating status of the system.

The system 900 also can be equipped with a counting device that counts or records the number of doses administered and the amount of each dose. In one implementation, for example, the controller 960 can have memory for recording dose information (e.g., number and amount of each dose) and other information regarding the operation of the system. Information recorded in the memory can be displayed on the digital readout 966. The device 900 also can include a removable memory device (e.g., a flash memory card) for storing such operating information. Additionally, a communication port (not shown) can be provided to allow operating information of the device 900 to be communicated to a general purpose computer (e.g., a laptop, desktop, hand-held, smartphone, etc.) via a cable or a wireless or other connection.

Also provided herein is a removable aerosolizing device for use with an aerosol delivery device. The removable aerosolizing device comprises an aerosolizing element and a source of agent. As described below, the source of agent can be configured to be selectively placed into fluid communication with the aerosolizing element to deliver agent contained therein to the aerosolizing element and the aerosolizing element is configured to be energized or actuated to generate an aerosol from the delivered agent. The generated aerosol can be used for inhalational administration to a subject.

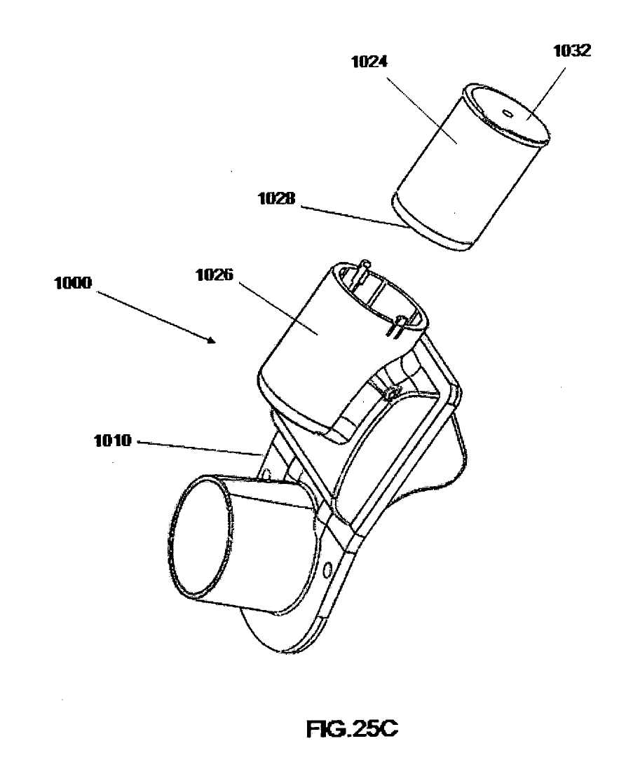

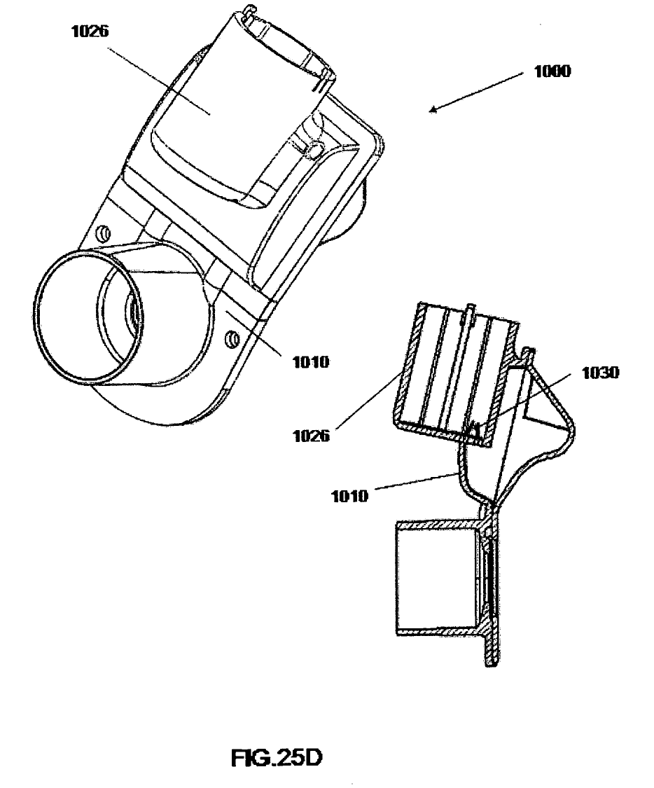

An aerosolizing element comprises means for producing an aerosol from agent delivered to the aerosolizing element. In one aspect, the aerosolizing element comprises a biasable mesh element. An example of a biasable mesh 1018 is shown in FIG. 25A. In another aspect, the aerosolizing element comprises a biasable plate element. An example of a biasable plate 1008 is shown in FIG. 25A. In yet another aspect, the aerosolizing element comprises a biasable plate element 1008 and a biasable mesh element 1018. One or more biasable elements can be in communication with an energy source or acutator. Application of energy or a force to the biasable elements from the energy source can cause one or more biasable element to bias and to produce an aerosol. In one example, ultrasound energy is applied to a biasable element to produce an aerosol.

If a mesh element and a plate element are used, a portion of the biasable plate element can operatively contact a portion of the biasable mesh element and a space can be defined between the biasable plate and the biasable mesh element. The source of agent is configured to be selectively placed into fluid communication with the space between the biasable plate and the biasable mesh element and, as one skilled in the art will appreciate, the aerosolizing element is configured to generate an aerosol from agent delivered into the space between the biasable plate and the biasable mesh element.

In one aspect, the removable aerosolizing device can further comprise a housing and the aerosolizing element and source of agent can be operatively attached to the housing. In one aspect, the source of agent can be configured to be constrained by the housing to prevent complete removal of the source of agent therefrom the housing.

Also provided herein is an aerosol delivery device that can, in one aspect, comprise means for delivering an aerosol when a patient inhales. For example, an aerosol delivery device can comprise a housing or body and a removable aerosolizing device comprising an aerosolizing element and a source of agent operatively attached thereto the housing or body. When the disposable aerosolizing element is operatively attached to the aerosol delivery device, the aerosolizing element is in fluid communication with the source of agent and is configured to be energized or actuated to aerosolize agent delivered therefrom the source of agent. The aerosol delivery device can also comprise a patient interface in fluid communication with the aerosolizing element that is configured to direct aerosolized agent to a subject upon inhalation by the subject.

FIGS. 25A and 25B show an exemplary embodiment of a removable aerosolizing device for use with an aerosol delivery device that can be exemplarily used to deliver aerosolized compositions to a subject for inhalation. FIG. 25A shows an exploded view of the removable aerosolizing device. FIG. 25B shows a perspective view of the removable aerosolizing device. The aerosolizing device 1000 comprises a body 1010 having an inner surface 1011 and defining an exit port 1013. In one aspect, the body 1010 can comprise a back housing portion 1004 and a front housing portion 1005.

Optionally, the removable aerosolizing device 1000 can further comprise acoustic coupling layer 1002 (such as tape, or a comparable adhesive, connector or laminar coupling mechanism) which is sized to fit through an aperture 1006 in the back housing portion 1004. In this aspect, the acoustic coupling layer is configured to engage a biasable element and a means for actuating or biasing the biasable element.

The removable disposable aerosolizing device can comprise an aerosolizing element. The aerosolizing element can comprise a biasable element 1008 having an inner surface 1007 and an opposed outer surface 1009. In one example, the biasable element is a biasable plate. A portion of the inner surfaces of the body 1011 and the biasable element 1007 define an interior space. In one aspect, the biasable element 1008 can be positioned such that its outer surface 1009 is contacted by the acoustic coupling tape 1002.

The acoustic coupling tape can be double-sided tape such that one side 1001 of the double-sided tape contacts and adheres to the outer surface 1009 of the biasable element whereas the opposite side 1003 of the acoustic coupling tape contacts an ultrasonic horn, transducer, actuator, or portion thereof of the aerosolizing device. In another aspect, the removable aerosolizing device can further comprise a single-sided adhesive element 1016 positioned between the biasable element 1008 and the front housing portion 1005.

Both the biasable element 1008 and the single-sided adhesive element 1016 can be sized and shaped to fit into respective cavities 1012 and 1020 within the front housing portion 1010. In one aspect, the back housing portion 1004 can be ultrasonically welded in approximation to the front housing portion 1010 as shown in FIG. 25B. Optionally, the back housing portion 1004 can also be adhered to the front housing portion 1010 using an optional adhesive element 1014.

When the back housing portion 1004 is brought into approximation with the front housing portion 1010, the biasable element 1008 and single-sided tape element 1016 are positioned between the front housing portion and the back housing portion. Moreover, the front surface 1001 of the double-sided acoustic coupling tape 1002 is positioned against the outer surface 1009 of the biasable element 1008 such that the aperture or hole 1006 in the back housing is positioned over the acoustic coupling tape. The ultrasonic horn, energizing element or actuator can contact the back surface 1003 of the acoustic coupling tape 1002 through the aperture 1006 in the back housing portion 1004 when the aerosolizing system or device is in operation or when the removable aerosolizing device is operatively positioned within the device.

In one exemplary aspect, the single-sided tape element 1016 has a single adhesive side that faces away from the inner surface 1007 of the biasable element and towards the exit port 1013. The single-sided tape element can be ring shaped. The diameter of the adhesive side can be less than the diameter of the exit port 1013. Aerosolized compositions are passed there through the aperture of the single sided tape element ring 1016 and through the exit port 1013 for delivery into the pulmonary system of the subject.

The removable aerosolizing device can further comprise a mesh element 1018 having an inner surface 1019. In one example, the mesh element is also biasable. A portion of the inner surface of the mesh element can define a portion of the interior space. Because the single-sided adhesive tape has an adhesive surface of a diameter that is smaller than the diameter of the exit port, a portion of the adhesive layer is exposed to the inner surface 1019 of the mesh 1018. Thus, the mesh can be adhered to the single-sided tape layer. The mesh can comprise one or more orifices and can be positioned such that agent is expelled through the one or more orifices of the mesh element. The mesh can be made of a variety of materials. For example, in one preferred embodiment, the mesh is made from gold. In some aspects, the mesh is biasable and can be biased by the horn or energizing element.

Thus, in operation, the back housing portion 1004 is welded, adhered using the optional adhesive layer 1014, or otherwise conventionally connected to the front housing portion 1010. The biasable element or plate 1008 and single sided tape element are positioned between the front and back housing portions. On the outer surface 1003 of the biasable element 1008 is adhered the double-sided acoustic tape layer 1002, if used, which is operatively positioned such that the back surface 1003 of the double-sided tape layer is accessible through the hole 1006 in the back housing portion such that ultrasonic energy can be delivered to the acoustic coupling tape from an energizing means located therein the aerosol delivery device. In one aspect, the energizing means comprises an ultrasonic transducer. Energy is transferred through the tape element 1002 and into the biasable element 1008 causing biasing of the inner surface 1007 of the biasable element 1008. The movement or biasing of the inner surface 1007 causes agent located in the interior space to be aerosolized. The aerosolized agent can be expelled through the exit port 1013 and mesh 1018.

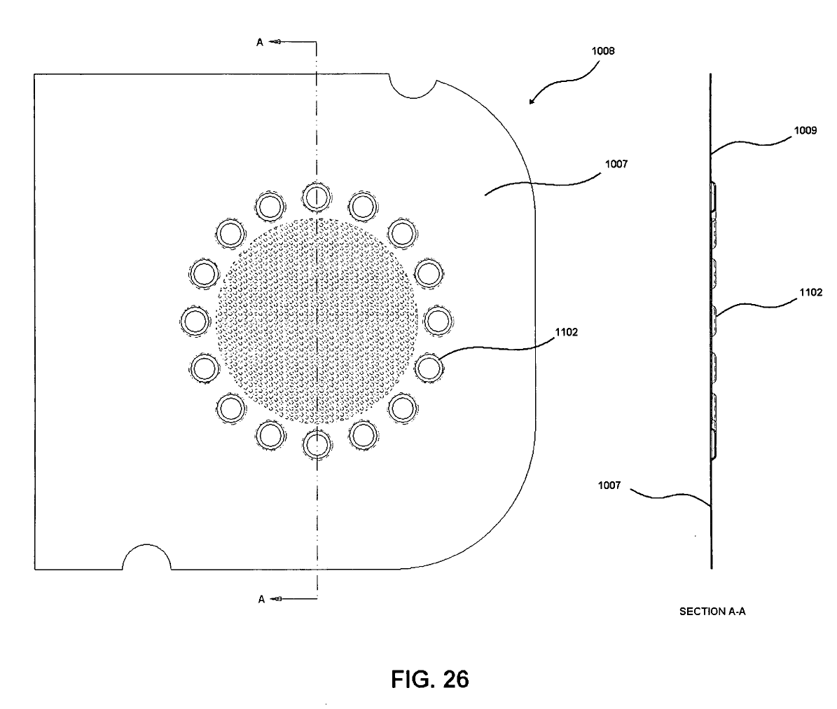

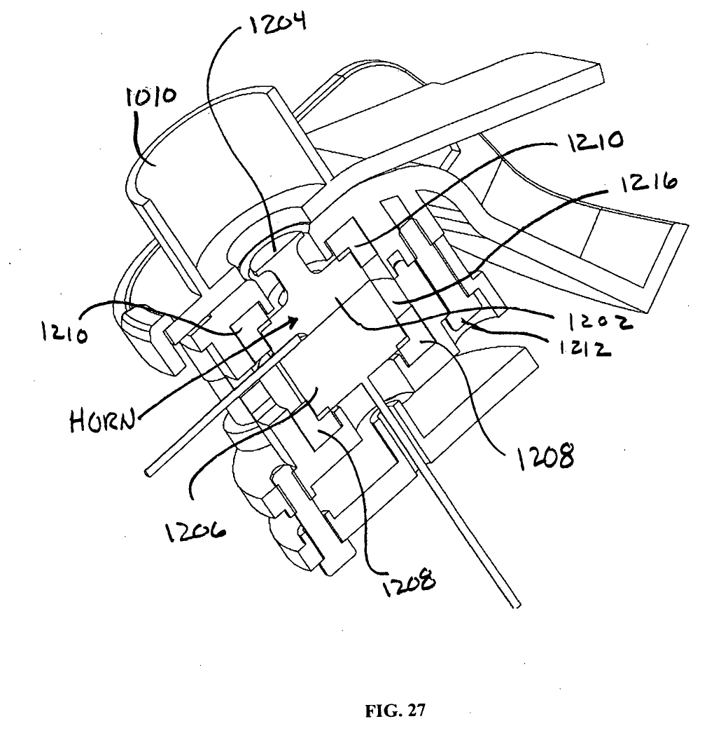

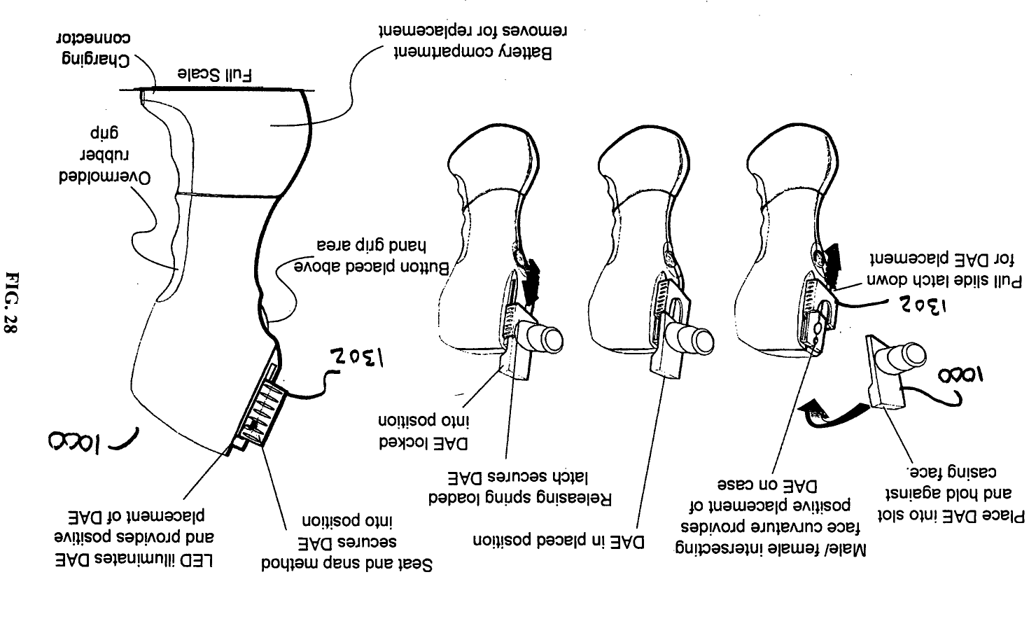

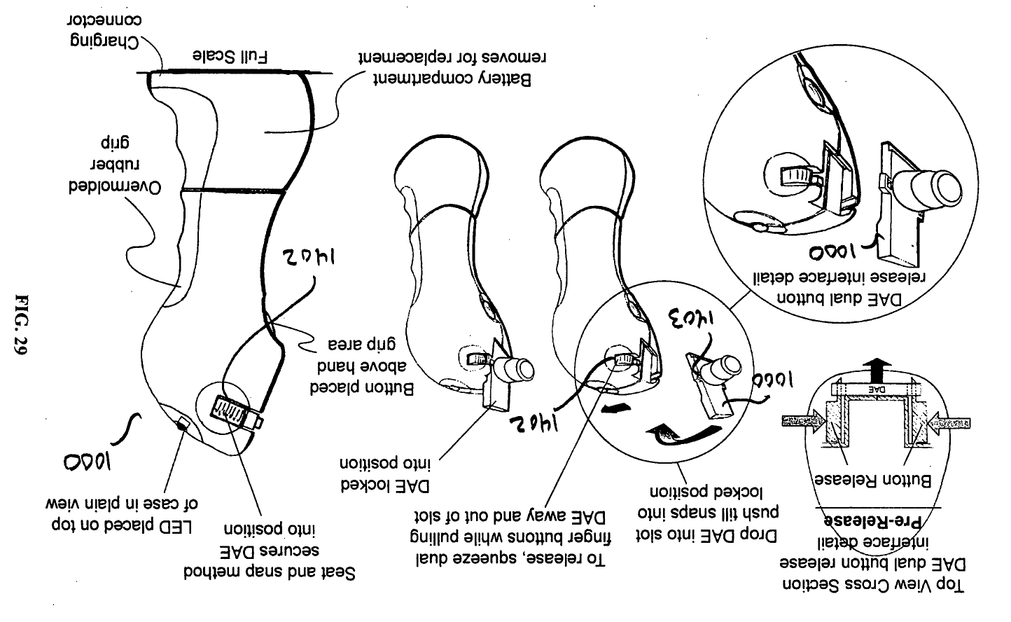

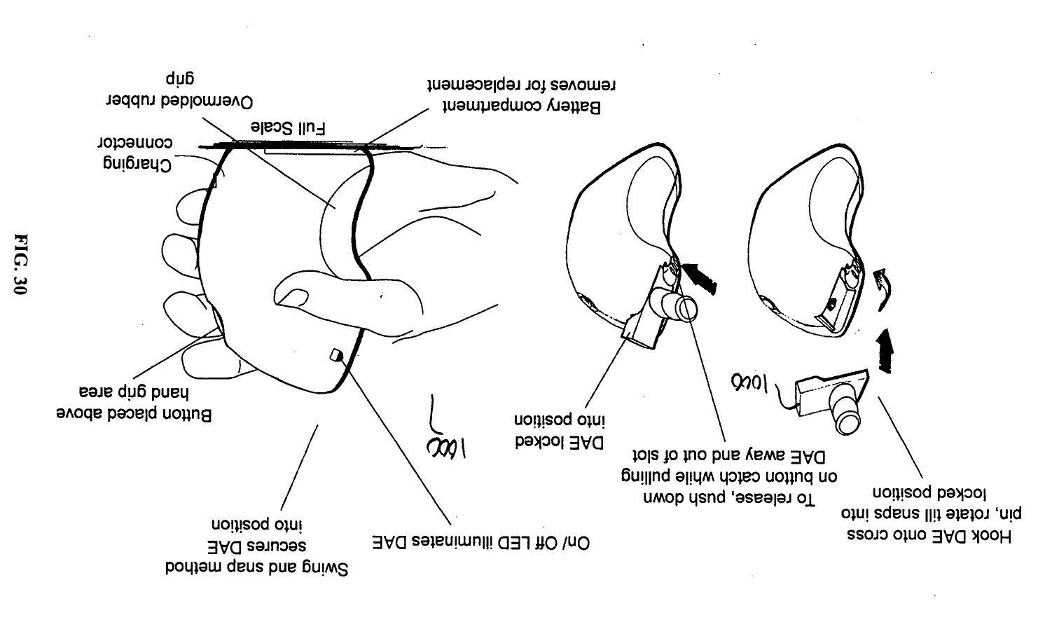

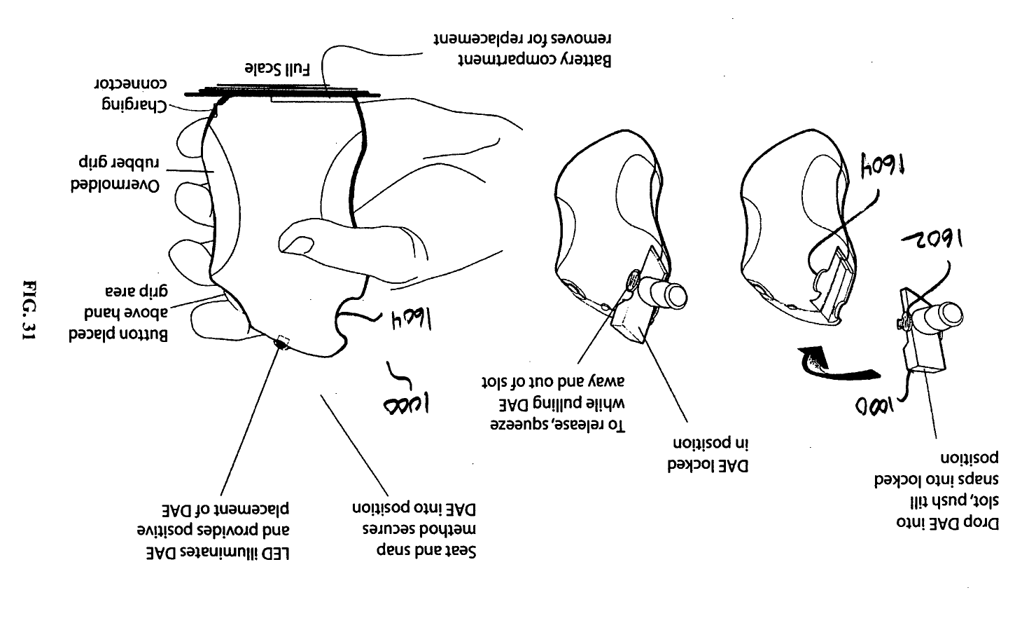

Fluid or agent to be aerosolized can be located between the inner surface of the biasable element and the mesh 1018. In this regard, the inner surface of the body 1011, the inner surface 1007 of the biasable element, and the inner surface 1019 of the mesh element define an interior space wherein fluid or agent is aerosolized by the transferred aerosolizing energy. From this space, the aerosolized agent is expelled through the exit port 1013 and mesh 1018. The biasing element 1008 can comprise one or more protrusions 1102 on its inner surface 1007 as shown in FIG. 26. The protrusions can contact a portion of the inner surface 1019 of the mesh 1018. Fluid or agent can accumulate between the biasing element or plate and the mesh.

Fluid or agent can be delivered to the interior space defined by the inner surfaces of the mesh, the biasable element and the body from a cartridge or vial 1024. The cartridge or vial 1024 can comprise a first surface 1028 and a second surface 1032 wherein the first surface 1028 can be penetrable. The second surface 1032 can be covered with a foil or vent tape which can be removed in operation to improve flow of fluid or agent into the interior space.

The cartridge or vial 1024 can be sized to be received within a receptacle 1026 defined by the front housing portion 1010 of the body. In a preferred embodiment, the cartridge 1024 is confined within the receptacle 1026 such that it cannot be easily removed. In this configuration, the cartridge 1024 can be pre-filled with a pre-determined volume of a composition comprising an agent. The cartridge can be located therein the receptacle without the first surface 1028 having been penetrated to release any fluid or agent. In this configuration, as shown in FIG. 25D, the receptacle 1026 can comprise a piercing element 1030 for piercing the first side 1028 of the cartridge to release fluid into the interior space. For this piercing to occur, the cartridge can be selectively moved within the receptacle and onto the piercing element.

In one preferred embodiment, a spacer can be selectively removed to allowing for the cartridge to be moved towards the piercing element and to be pierced by the piercing element. In another aspect, the cartridge or vial can comprise a piercing element. In this aspect, the receptacle portion can comprise a penetrable surface and the cartridge can be selectively moved such that the piercing element pierces the penetrable surface of the cartridge to allow flow of fluid into the inner surface.

The second surface of the cartridge 1032 can also be vented to enhance fluid flow from the cartridge to the interior space. In one preferred embodiment, the back surface 1032 is porous but is covered with a venting tape that can be selectively removed when venting is desired. In this embodiment, a user can move the cartridge onto the piercing element to affect piercing of first penetrable surface of the cartridge and subsequently remove the vent tape to enhance the fluid flow into the interior space. As shown in FIG. 25, the back housing portion 1004 can comprise a reservoir 1034 for receiving fluid from the pierced cartridge which can then be delivered by gravity or by capillary action to the active area between the biasable element 108 and the mesh 1019.

The cartridge or vial 1024 can hold a variety of compositions in a variety of volumes. For example, in some preferred embodiments, the cartridge can hold between 50 microliters and 10 milliliters of fluid. The cartridge can be sized to hold a pre-determined volume of fluid and can also be sized to fit within the receptacle of the front housing portion 1026. Thus, each pre-determined volume cartridge can be located within the receptacle of the front housing such that the removable aerosolizing device can be fully removed from the aerosolizing device with the receptacle and cartridge intact.

The acoustic coupling tape layer 1002 attached to the outer surface 1009 of the biasable element 1008 can comprise a foil or covering layer for covering the its back adhesive side 1003. The covering layer is configured to cover the adhesive that comes into contact with the ultrasonic horn when the removable aerosolizing device is placed into the aerosol delivery device. In operation, the covering layer can be removed prior to the removable aerosolizing element being seated in the aerosol delivery device to allow the ultrasonic horn to contact the adhesive layer.

When the removable aerosolizing device is operatively seated into the aerosolizing device, a spring-loaded force can be used to force the ultrasonic horn into communication with a portion of the removable aerosolizing element. Such a force can be used to establish and maintain contact between the acoustic coupling tape 1002 and the horn.

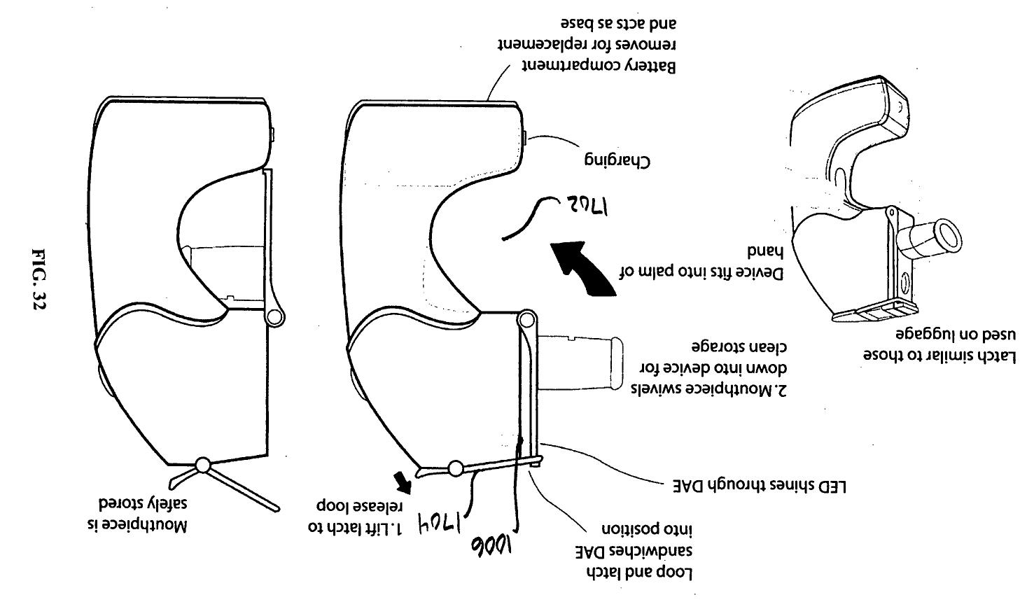

The biasable element 1008 and/or the mesh 1019 can be biased by an applied energy or force. In one example, ultrasonic energy is applied to bias the element 1008 and/or the mesh 1019. FIG. 27 shows an ultrasonic horn 1202 which can be used to bias the element 1008 and/or the mesh 1019. The horn comprises a portion 1204 which contacts and adheres to the back side of the acoustic coupling tape 1002. The acoustic horn also comprises a portion which overlies and is in operative communication with an ultrasonic transducer 1206. The horn can be forced into communication with the coupling tape by a spring mechanism underlying the transducer and/or horn. Furthermore, a resilient or elastomeric gasket 1208 can be used to provide a force for pressing the portion of the horn 1204 against the acoustic coupling tape 1002.

For example, an annular gasket 1208 can be used to provide the force to press or urge the horn against the acoustic coupling tape. In one embodiment, an angular gasket can be used. Such a gasket can be made of material known to those skilled in the art such as rubber. A gasket can be positioned below the PZT transducer such that a force from the gasket into the transducer is transferred into the horn 1202 to urge the horn into the acoustic coupling tape. In another embodiment, a spring can provide the urging force to the transducer and horn. The spring can be positioned anywhere behind the PZT transducer or on the side of the PZT transducer in contact with the ultrasonic horn.

In some embodiments, a sealing gasket 1210 can also be used. The sealing gasket 1210 can be used to seal the horn to the face of the device. A screw 1212 can used to adjust the compression on the sealing gasket 1210. Similarly, a screw 1214 can be used to adjust the compression on the gasket 1208 or on the compression of the spring not shown.

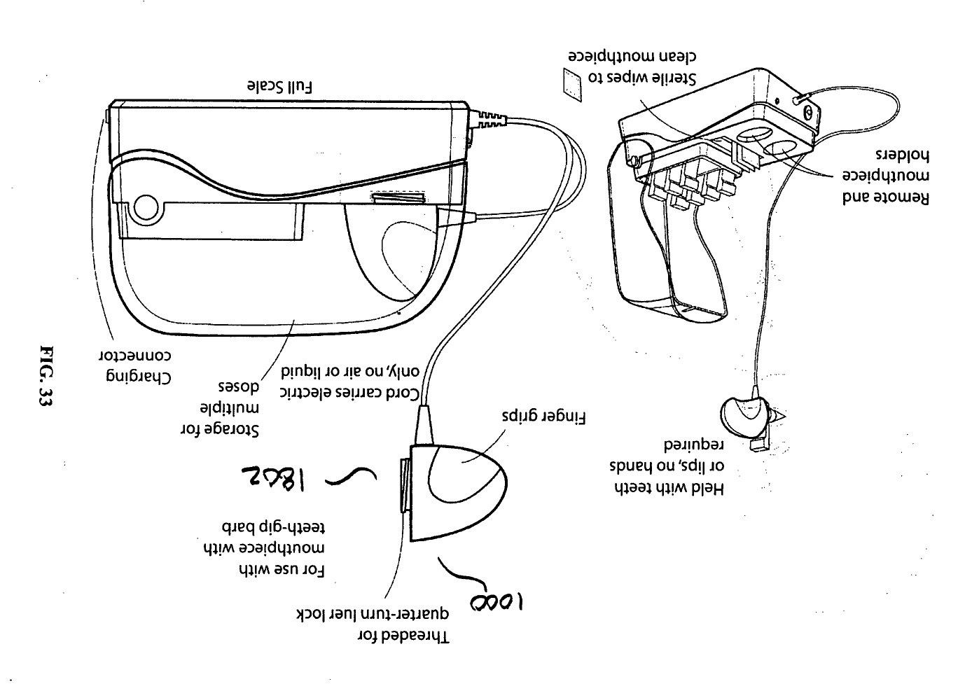

The portion of the horn that contacts the acoustic coupling tape 1002 can take a variety of shapes. For example, it can be concave or convex. In one preferred embodiment, the shape is convex such that it protrudes towards back surface 1003 of the acoustic coupling tape 1002. FIG. 28 shows a slideable element 1302 that can be selectively moved over a rebiasable aerosolizing device 1000 to secure the rebiasable aerosolizing device to the aerosol delivery device. The slideable element can also be moved off of the disposable aerosolizing device to allow the removal of the removable aerosolizing device 1000 from the hand-held aerosolizing device for operative use.

An aerosol delivery device can comprise means for delivering an aerosol when a patient inhales. For example, an aerosol delivery device can comprise a housing and a removable aerosolizing device comprising an aerosolizing element and a source of agent operatively attached thereto the housing. The aerosolizing element is in fluid communication with the source of agent and is configured to be energized to aerosolize agent delivered therefrom the source of agent. The aerosol delivery device can further comprise a patient interface in fluid communication with the aerosolizing element that is configured to direct aerosolized agent to a subject upon inhalation by the subject.

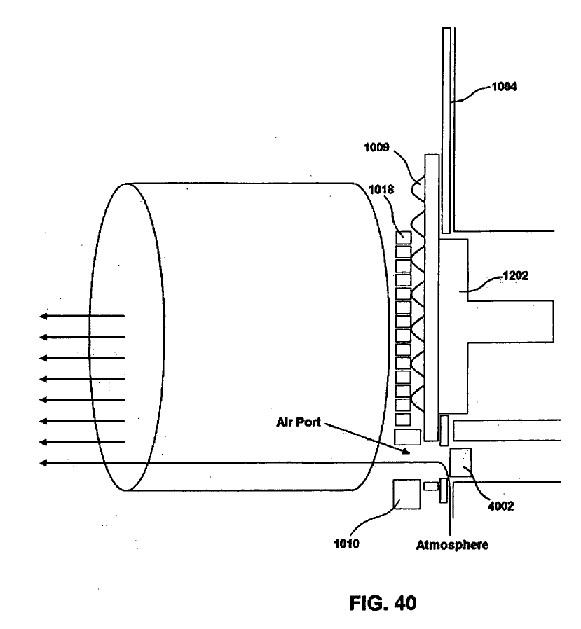

In one aspect the aerosol delivery device comprise a feedback means for energizing or actuating the aerosolizing element to aerosolize the delivered agent. As shown in FIG. 40, the feedback means can comprise pressure detecting means 4002 for detecting a pressure change based on inhalation of the subject. The pressure detecting means can located in at least one of the housing, removable aerosolizing device, and patient interface. The feedback means can further comprise a switchable actuator in operative communication with the pressure detecting means and is configured to be switched between an active and deactivate state, wherein the active state energizes the aerosolizing element causing aerosolization of the agent.

In one aspect, it is contemplated that the switching means can be configured to activate the actuator when the detected pressure meets or is lower than a predetermined pressure value. In this aspect, the switching means can also be configured to deactivate the actuator when the detected pressure exceeds a predetermined pressure value.

FIG. 29 shows exemplary means for securing a removable aerosolizing device 1000 to the hand-held aerosolizing device. The removable aerosolizing device 1000 can comprise receiving tabs 1403 which can be snapped into approximation with projections 1402 on the hand-held device to secure the removable aerosolizing device to the aerosol delivery device.

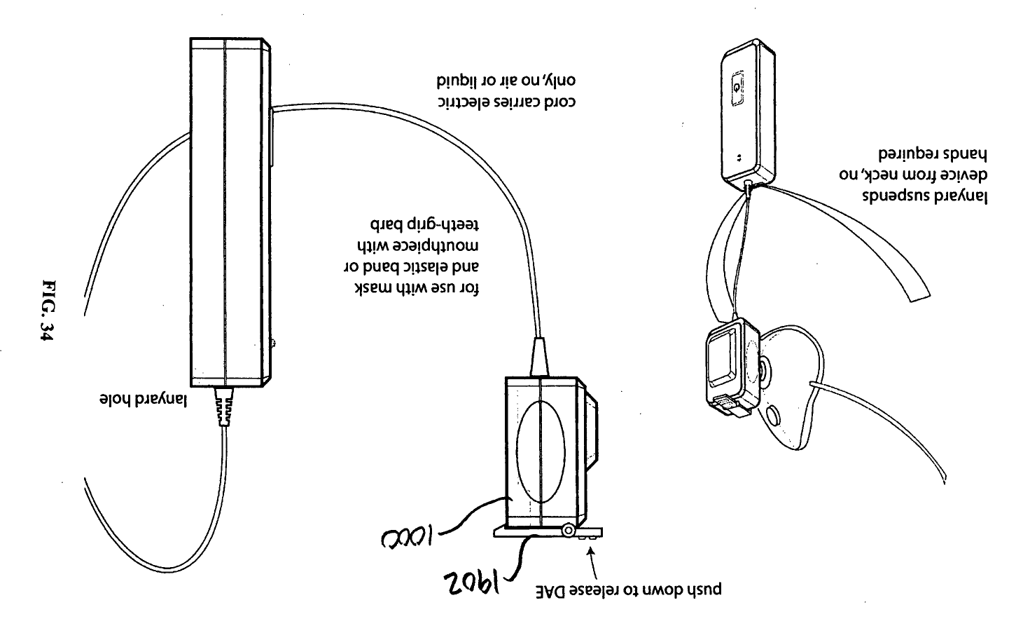

FIG. 30 shows yet another embodiment for securing a removable aerosolizing device 1000 to a hand-held aerosol delivery device. The removable aerosolizing device 1000 can be positioned against the hand-held device and a tab 1502 can be moved up or down to temporarily secure the removable aerosolizing device 1000 into position.

FIG. 31 shows yet another embodiment for temporarily securing the removable aerosolizing device 1000 to an aerosol delivery device. The removable aerosolizing device 1000 can be constructed with two protruding tabs 1602 which can be mated with complimentary receptacles 1604 for securing removable aerosolizing device 1000 into position on the aerosol delivery device. The removable aerosolizing device 1000 can be released by medially squeezing the tabs 1602 while pulling the removable aerosolizing device 1000 out and away from the aerosol delivery device. Thus the removable aerosolizing device 1000 can be snapped and seated into the aerosol delivery device and secured into position.

FIG. 32 shows yet another embodiment for temporarily securing the removable aerosolizing device 1000 into a hand-held aerosol delivery device. In this embodiment, the removable aerosolizing device 1000 is positioned in a receiving portion of the device. A latch portion 1702 is pivoted over the removable aerosolizing device 1000 which is latched into position with a latching mechanism 1704 for securing the removable aerosolizing device into position.

FIG. 33 shows yet another embodiment of a removable aerosolizing device 1000. In this embodiment, the removable aerosolizing device 1000 is secured with a threaded mechanism common to those skilled in the art.

FIG. 34 shows yet another embodiment of a removable aerosolizing device 1000 and aerosol delivery device. In this embodiment, the removable aerosolizing device 1000 is temporarily secured to the aerosol delivery device using a pivotable latching mechanism 1902.

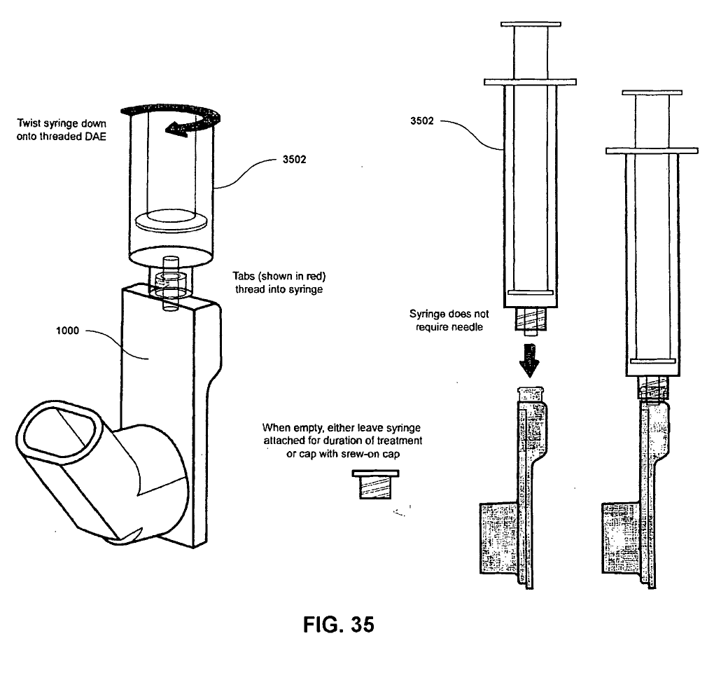

FIGS. 35 through 39 show alternate ways to attach an agent container or source to the removable aerosolizing device 1000.

FIG. 35 shows a syringe-type mechanism 3502 that is screwed into a receiving portion of the removable aerosolizing device. Typical screw connections between a syringe and a receptacle are commonly known in the art. In this embodiment fluid contained in the syringe can be injected or delivered into the removable aerosolizing device. After delivery, the syringe can be unscrewed and removed from the removable aerosolizing device leaving the removable aerosolized device filled for aerosolization.

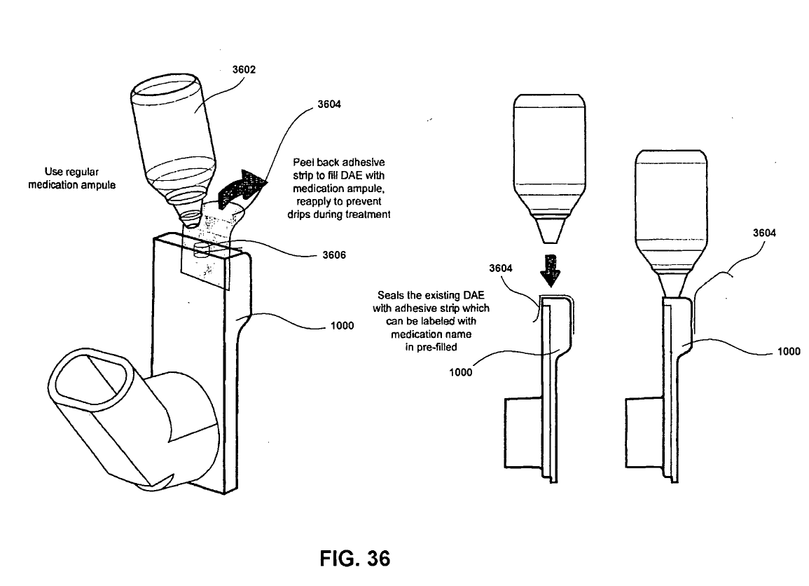

FIG. 36 shows another embodiment for delivering fluid into the removable aerosolizing device using a medication ampule or vial 3602. In this embodiment the removable aerosolizing device 1000 is equipped with a peel-back adhesive or foil layer 3604 which can be temporarily displaced to allow inlet hole 3606 to mate with the ampule 3602 for delivery there through the hole into the removable aerosolizing device 1000. After a pre-determined amount of fluid is delivered into the removable aerosolizing device 1000, the ampule 3602 can be removed and the foil or adhesive can be replaced over the hole 3606 to prevent escape of fluid through said hole or to prevent contamination of the removable aerosolizing device.

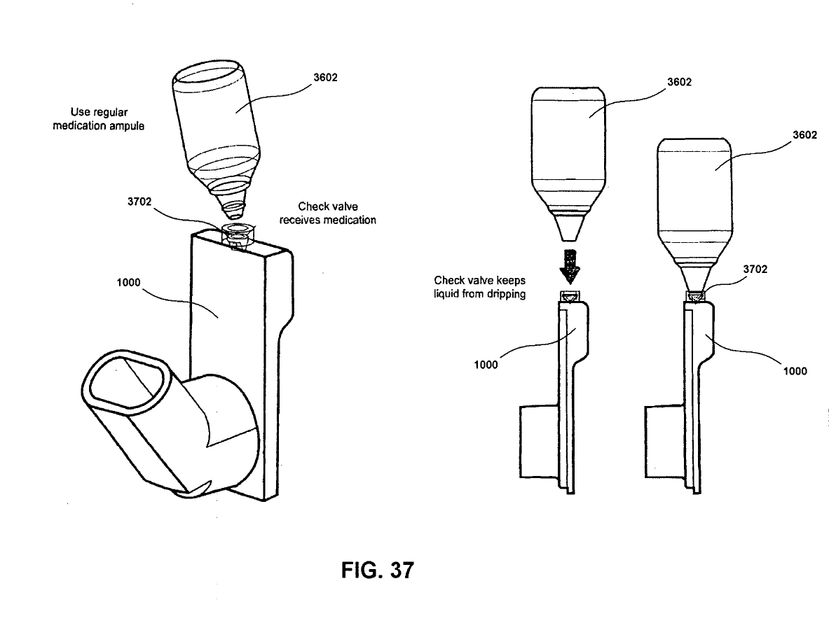

FIG. 37 shows yet another embodiment for filling the removable aerosolizing device 1000 using an ampule or vial of fluid or medication. In this embodiment, the aerosolizing element comprises a check valve wherein the fluid can be delivered from the ampule in a one-way direction through the valve and into the removable aerosolizing device. Thus, the valve can prevent backflow into the ampule.

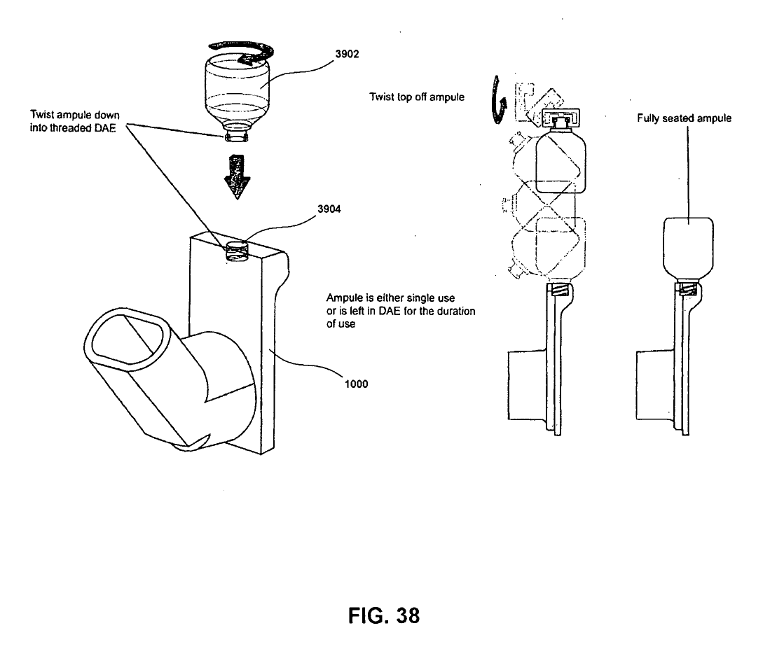

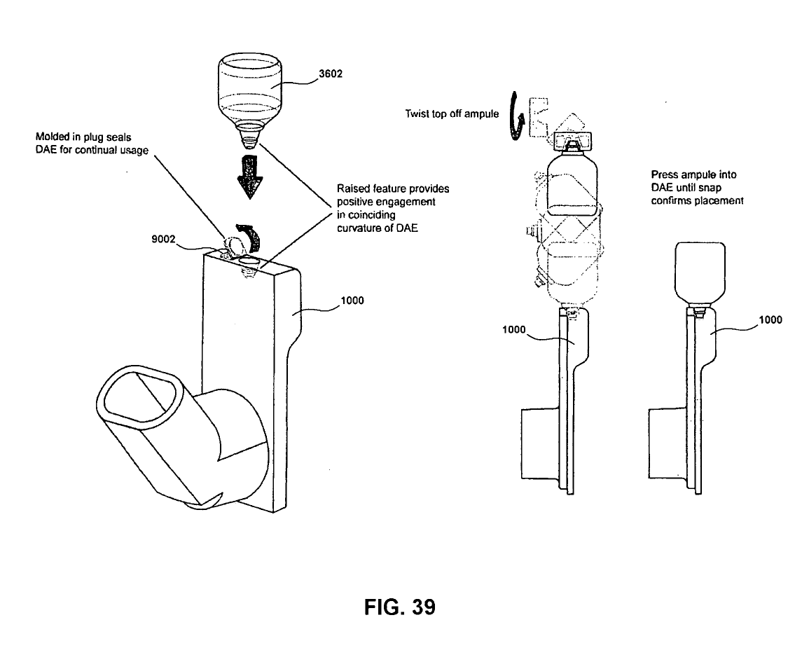

FIG. 38 shows yet another embodiment for connection of a vial or ampule to the removable aerosolizing device 1000. In this embodiment, a threaded ampule or vial is used, which can be threaded onto a corresponding threaded portion 3904 of the removable aerosolizing device 1000. The ampule can be threaded into place for delivery of fluid into the removable aerosolizing device.

FIG. 39 shows yet another embodiment for connecting an ampule 3602 to the removable aerosolizing device 1000 for delivery of fluid or medication into the removable aerosolizing device. In this embodiment, a plug 9002 can be positioned on the removable aerosolizing device for securing or plugging the hole that is designed to mate with the ampule for delivery of fluid into the removable aerosolizing device. After fluid is delivered and the ampule is removed, the plug can be placed back into the hole to seal the fluid therein the removable aerosolizing device.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

What is claimed is:

1. A removable aerosolizing device for use with an aerosol delivery device, comprising:
a body having an inner surface and defining an exit port;
a biasable element having an inner surface and an opposed outer surface, wherein a portion of the respective inner surfaces of the body and the biasable element define an interior space; and
a source of agent configured to be selectively placed into fluid communication with the interior space, wherein the biasable element is configured to be biased in response to a force applied to the outer surface to expel agent in the interior space through the exit port.

2. The removable aerosolizing device of claim 1, further comprising a mesh element having an inner surface, wherein a portion of the respective inner surface of the mesh element defines a portion of the interior space, wherein the mesh element comprises one or more orifices, and wherein the mesh element is configured to be positioned therein at least a portion of the exit port such that the agent is expelled through the one or more orifices of the mesh element.

3. The removable aerosolizing device of claim 1, wherein the source of agent is positioned in a receptacle defined by the body.

4. The removable aerosolizing device of claim 3, wherein the source of agent is selectively movable therein the receptacle.

5. The removable aerosolizing device of claim 4, wherein the source of agent is constrained within the receptacle to prevent complete removal of the source of agent therefrom the receptacle.

6. The removable aerosolizing device of claim 3, wherein the receptacle comprises a piercing element for piercing at least a portion of the source of agent.

7. The removable aerosolizing device of claim 6, wherein the piercing element is configured to be in fluid communication with the interior space so that the agent can selectively flow out of the source of agent and into the interior space.

8. The removable aerosolizing device of claim 7, wherein the source of agent can be selectively moved onto the piercing element to affect the piercing.

9. The removable aerosolizing device of claim 8, wherein a portion of the exterior surface of the source of agent is configured to slideably move therein a pathway defined in a portion of the receptacle.

10. The removable aerosolizing device of claim 8, wherein the source of agent comprises a first surface that can be penetrated by the piercing element.

11. The removable aerosolizing device of claim 10, further comprising a spacer element positioned between the piercing element and the first surface of the source of agent, wherein the spacer element is removable to allow selective movement of the source of agent onto the piercing element to affect the piercing.

12. The removable aerosolizing device of claim 10, wherein the receptacle further comprising means for orienting the first surface of the source of agent relative to the piercing element as the source of agent is slideably moved onto the piercing element.

13. The removable aerosolizing element of claim 10, wherein the source of agent further comprises a second surface that can be selectively vented to facilitate flow of agent into the space.

14. The removable aerosolizing element of claim 13, wherein the second surface of the source of agent opposes the first surface of the source of agent.

15. An aerosol delivery device, comprising:
a housing;
a removable aerosolizing device that comprises a biasable portion and that defines an interior space, wherein the removable aerosolizing device is operatively attached thereto the housing;
an agent disposed within the interior space of the removable aerosolizing device;
an actuator disposed in the housing configured to be positioned in forced communication with a portion of the removable aerosolizing device, wherein the actuator comprises means for exerting a force on the biasable portion of the removable aerosolizing device to aerosolize agent in the interior space; and
a patient interface in fluid communication with the interior space that is configured to direct aerosolized agent to a subject.

16. The aerosol delivery device of claim 15, further comprising a spring mechanism disposed in the housing and selectively positioned to force the actuator into communication with at least a portion of the biasable portion of the aerosolizing device.

17. The aerosol delivery device of claim 16, further comprising an elastomeric element disposed in the housing and selectively positioned to force the actuator into communication with at least a portion of the biasable portion of the aerosolizing device.

18. The aerosol delivery device of claim, 15, wherein the actuator comprises an ultrasonic transducer and an ultrasonic horn.

19. The aerosol delivery device of claim 18, wherein the ultrasonic horn is convex in the direction of the biasable portion.

20. A removable aerosolizing device for use with an aerosol delivery device, comprising:
an aerosolizing element; and
a source of agent configured to be selectively placed into fluid communication with the aerosolizing element to deliver agent contained therein to the aerosolizing element, wherein the aerosolizing element is configured to be energized to generate an aerosol from the delivered agent for inhalational administration to a subject.

21. The removable aerosolizing device of claim 20, wherein the aerosolizing element comprises a biasable mesh element.

22. The removable aerosolizing device of claim 20, wherein the aerosolizing element comprises a biasable plate element.

23. The removable aerosolizing device of claim 20, wherein the aerosolizing element comprises a biasable plate element and a biasable mesh element.

24. The removable aerosolizing device of claim 23, wherein a portion of the biasable plate element operatively contacts a portion of the biasable mesh element.

25. The removable aerosolizing device of claim 24, wherein a space is defined between the biasable plate and the biasable mesh element.

26. The removable aerosolizing device of claim 25, wherein the source of agent is configured to be selectively placed into fluid communication with the space between the biasable plate and the biasable mesh element.

27. The removable aerosolizing device of claim 26, wherein the aerosolizing element is configured to generate an aerosol from agent delivered into the space between the biasable plate and the biasable mesh element.

28. The removable aerosolizing device of claim 20, further comprising a housing and wherein the aerosolizing element and source of agent are operatively attached to the housing.

29. The removable aerosolizing device of claim 28, wherein the source of agent is constrained by the housing to prevent complete removal of the source of agent therefrom the housing.

30. The removable aerosolizing device of claim 20, wherein the aerosolizing element is configured to be energized by ultrasonic energy.

31. An aerosol delivery device, comprising:
a housing;
a removable aerosolizing device comprising an aerosolizing element and a source of agent operatively attached thereto the housing, wherein the aerosolizing element is in fluid communication with the source of agent and is configured to be energized to aerosolize agent delivered therefrom the source of agent;
a patient interface in fluid communication with the aerosolizing element that is configured to direct aerosolized agent to a subject upon inhalation by the subject; and
feedback means for energizing the aerosolizing element to aerosolize the delivered agent.

32. The aerosol delivery device of claim 31, where in the feedback means comprises pressure detecting means for detecting a pressure change based on inhalation of the subject.

33. The aerosol delivery device of claim 32, wherein the pressure detecting means is located in at least one of the housing, removable aerosolizing device, and patient interface.

34. The aerosol delivery device of claim 33, wherein the feedback means further comprises a switchable actuator in operative communication with the pressure detecting means and configured to be switched between an active and deactivate state, wherein the active state energizes the aerosolizing element causing aerosolization of the agent.

35. The aerosol delivery device of claim 34, wherein the switching means is configured to activate the actuator when the detected pressure meets or is lower than a predetermined pressure value.

36. The aerosol delivery device of claim 34, wherein the switching means is configured to deactivate the actuator when the detected pressure exceeds than a predetermined pressure value.

37. A removable aerosolizing device for use with an aerosol delivery device, comprising:
a body having an inner surface and defining an exit port;
an aerosolizing element having an inner surface and an opposed outer surface, wherein a portion of the respective inner surfaces of the body and the aerosolizing element define an interior space; and
a source of agent configured to be selectively placed into fluid communication with the interior space, wherein the aerosolizing element is configured to be energized to expel agent in the interior space through the exit port.

38. The removable aerosolizing device of claim 37, wherein the aerosolizing element comprises a mesh portion having one or more orifices, and wherein the mesh element is configured to be positioned therein at least a portion of the exit port such that the agent is expelled through the one or more orifices of the mesh portion.

39. A removable aerosolizing device for use with an aerosol delivery device, comprising:
an aerosolizing element; and
a source of agent configured to be selectively placed into fluid communication with the aerosolizing element to deliver agent contained therein to the aerosolizing element, wherein the aerosolizing element is configured to be energized to generate an aerosol from the delivered agent.

40. The removable aerosolizing device of claim 39, wherein the aerosolizing element comprises a biasable mesh element.

41. The removable aerosolizing device of claim 39, wherein the aerosolizing element comprises a biasable plate element.

42. The removable aerosolizing device of claim 39, wherein the aerosolizing element comprises a biasable plate element and a biasable mesh element.

43. The removable aerosolizing device of claim 42, wherein a portion of the biasable plate element operatively contacts a portion of the biasable mesh element.

44. The removable aerosolizing device of claim 43, wherein a space is defined between the biasable plate and the biasable mesh element.

45. The removable aerosolizing device of claim 44, wherein the source of agent is configured to be selectively placed into fluid communication with the space between the biasable plate and the biasable mesh element.

46. The removable aerosolizing device of claim 45, wherein the aerosolizing element is configured to generate an aerosol from agent delivered into the space between the biasable plate and the biasable mesh element.

47. The removable aerosolizing device of claim 39, further comprising a housing and wherein the aerosolizing element and source of agent are operatively attached to the housing.

48. The removable aerosolizing device of claim 47, wherein the source of agent is constrained by the housing to prevent complete removal of the source of agent therefrom the housing.

49. The removable aerosolizing device of claim 39, wherein the aerosolizing element is configured to be energized by ultrasonic energy.
